(19) United States
(12) Patent Application Publication
(10) Pub. No.: US 2011/0300198 A1
(43) Pub. Date: Dec. 8, 2011

(54) HYDROCOLLOID - ESSENTIAL OIL PATCHES

(76) Inventors: Amos Nussinovitch, Rehovot (IL); Yulia Shcherbina, Rishon le-Zion (IL); Zvi Roth, Kvuzat-Shiller (IL)

(21) Appl. No.: 13/152,413

(22) Filed: Jun. 3, 2011

Related U.S. Application Data
(60) Provisional application No. 61/350,926, filed on Jun. 3, 2010.

(51) Int. Cl. A61K 8/02; A61K 36/53; A61K 9/70; A61Q 19/00; A61P 17/00; A61K 36/752; A61K 8/97

(52) U.S. Cl. 424/401; 424/736; 424/745; 424/443

(57) ABSTRACT

The present invention relates to dermal patches comprising natural polysaccharides without the need for any pressure sensitive synthetic polymers. Particularly, the present invention relates to patches comprising a bioadhesive composition comprising a polysaccharide exudate and an essential oil useful for transdermal delivery of therapeutic or cosmetic agents.

### CROSS-REFERENCE TO RELATED APPLICATIONS

This application claims the benefit of Provisional Patent Application No. 61/350,926 filed Jun. 3, 2010, the content of which is incorporated by reference herein in its entirety.

### FIELD OF THE INVENTION

The present invention relates to dermal patches having improved properties comprising natural polysaccharides and essential oils, preferably without the use of any synthetic adhesives. Particularly, the present invention relates to patches comprising a bioadhesive composition comprising a polysaccharide exudate useful for transdermal delivery of at least one essential oil as therapeutic or cosmetic agents.

### BACKGROUND OF THE INVENTION

Essential oils are volatile and liquid aroma compounds from natural sources, usually plants. Essential oils are not oils in a strict sense, but often share with oils a poor solubility in water. Essential oils are usually prepared by fragrance extraction techniques such as distillation (including steam distillation), cold pressing, or extraction (maceration). Typically, essential oils are highly complex mixtures of often hundreds of individual aroma compounds.

Essential oils are widely used in the food industry, in cosmetics and as pharmaceuticals. Dermal patches including essential oils that are released by evaporation or diffusion have been marketed, for instance as homeopathic remedies for symptoms of stress, menopause, various aches and pains or coughs and colds. However, most of these remedies are marketed under the heading of aroma therapy, which entails absorbance by inhalation and have not been shown to produce efficient transdermal transport of essential oils. For homeopathic remedies there is often little or no information on their production and/or physical and chemical properties. Essential oils have been used from ancient times for cosmetic or therapeutic uses. They historically and to the present day have been applied to the skin, inhaled or ingested by humans.

Pressure Sensitive Adhesives

Pressure-sensitive adhesives (PSAs) are adhesives that are capable of bonding to surfaces via brief contact under light pressure (Goulding, 1994). PSA's are an indispensable component of medicinal patches, medical devices, tapes, dressings and bioelectrodes. Several basic requirements must be fulfilled to provide an acceptable PSA product including (1) adequate skin adhesion and cohesion; (2) biocompatibility i.e. biologically inert, precluding contact dermatitis, allergy, sensitivity or toxicity; (3) repositioning ability on the skin surface for multiple applications; (4) small geometric dimensions; (5) reasonable cost; and (6) compliance with international pharmaceutical standards.

Elastomers are flexible polymer materials that function to increase the elasticity, tear resistance, and cohesiveness of adhesive compositions. Many of the known PSA elastomers cause physiological irritation including inflammation of sweat glands, keratin peeling, tissue injury after adhesive removal and contact dermatitis due to prolonged contact with the skin (Bergman et al., 1982; Hammond, 1989).

Three main types of polymers are commonly used in PSA dermatological products, particularly transdermal delivery (TDD) systems: polyisobutylenes (PIB), polysiloxanes (silicones) and polyacrylate copolymers (Tan and Pfister, 1999).

These polymers have several notable disadvantages. First, they are hydrophobic and retain only a small amount of moisture (<0.1%) after drying, thus limiting the type of active agents that can be incorporated and diminishing the electrical conductivity potential in iontophoresis. Moreover, the hydrophobic nature of the PSA prevents wick removal of accumulated moisture on the skin surface, increasing the risk of microbial infection. In addition, they are typically rigid, becoming soft and flexible only when their temperature exceeds the glass transition, posing problems in industrial manufacturing.

Hydrocolloids and Hydrogels

The art recognizes medicinal polymeric hydrocolloidal materials that are mucoadhesive, i.e. adhere to a subject's mucous membranes. In such applications, the dried hydrocolloids are applied to the mucosal tissue and tack occurs by swelling of the polymer by the biological fluids. Different chemo-physical factors affect mucoadhesive properties including type of polymer, its concentration and molecular weight (Chen and Cyr, 1970); viscosity of the polymer dispersion; matrix hydration capability; polymeric mixtures; polymer pH and electrical charge; adhesive-layer thickness; and shearing (Chen and Cyr, 1970).

Sterculia gum, also known as gum karaya, is a hydrophilic colloid prepared from the exudate of the Sterculia Urens tree. It is a complex polysaccharide gum comprised mainly of D-galacturonic acid, D-galactose and L-rhamnose, having a molecular weight of about 9-10×10⁶ Daltons.

U.S. Pat. No. 4,299,231 discloses an electrically conductive, visco-elastic gel comprising 10 to 50% of a high molecular weight polysaccharide such as karaya gum, 90 to 20% of at least one polyol, the polyol having a water content of 5 to 20% by weight, 0 to 30% of at least one non-volatile acid soluble in said polyol, 0 to 30% of at least one non-volatile base soluble in said polyol for use in adhering or producing medical electrodes. The preferred polysaccharides disclosed in U.S. Pat. No. 4,299,231 include gum karaya, gum tragacanth, xanthan gum, and carboxymethylcellulose. The gels are disclosed as having relatively low water content, which allows open-air storage.

U.S. Pat. No. 3,640,741 teaches a mixture of a hydrophilic gum and a cross-linking agent, such as propylene glycol, in a non water-soluble carrier, the mixture forming a gel, useful for providing for timed release of medication in the body or cosmetic additives on the surface of a person's skin. In one specific embodiment the hydrophilic gum comprises a mixture of carboxymethylcellulose or sodium alginate and karaya gum. According to U.S. Pat. No. 3,640,741, karaya gum should not be used to fully substitute the cellulose or alginate gums.

U.S. Pat. No. 4,306,551 teaches a flexible, liquid absorbable adhesive bandage comprising a backing and a substrate, the substrate comprising a solid phase comprising about 30%-50% by weight and a liquid phase of hydric alcohol, carbohydrates or proteins comprising about 50-70% by weight, further comprising a synthetic resin selected from polyacrylic acid, polyacrylamide and their congeners.

U.S. Pat. No. 4,307,717 teaches a flexible, liquid-absorbent, adhesive bandage comprising the matrix taught in the U.S. Pat. No. 4,306,551, further comprising a medicament for release to the surface to which the bandage is applied.

U.S. Pat. No. 4,778,786 teaches a gelation reaction product of a mixture of an organic polysaccharide gum, polyethylene glycol, and m-, p- or o-hydroxybenzoic acid in an amount effective in forming a gel having adhesive properties for adhesion to skin for transdermal drug delivery. The U.S. Pat. No. 4,778,786 teaches that polyethylene glycol and m-, p- or o-hydroxybenzoic acid combine with polysaccharide gums to form a gel having both desirable tackiness/deformability and desirable structural integrity whereas polyethylene glycol and polysaccharide gums, without m-, p- or o-hydroxybenzoic acid, mostly fail to form gels or form mushy gels lacking structural integrity even at modest concentrations of polyethylene glycol.

U.S. Pat. Nos. 5,536,263 and 5,741,510 teach a non-occlusive medication patch to be applied to the skin, the patch comprising a porous backing and a flexible hydrophilic pressure-sensitive adhesive reservoir comprising a hydrocolloidal gel for the sustained release of medication through the skin of a patient. The reservoir has two portions: an external coating layer with an exposed lower skin-contacting surface that forms a pressure-sensitive bond with the skin, and an upper internal portion which infiltrates the porous backing and becomes solidified therein after being applied so that the reservoir and the backing are unified, enabling the backing itself to act as a storage location for the medication-containing reservoir.

A previous patent application WO 2006/0085329 to one of the inventors of the present invention and others discloses hydrophilic compositions comprising polysaccharide-based hydrocolloid gum exudates modified by chemical or physical means to provide superior pressure sensitive adhesive (PSA) materials. The simplicity of the matrix, and ease of manufacture, provides a significant advantage over standard PSA materials. It is further disclosed that the modified polysaccharide-based hydrogels are particularly useful as depots for biologically active ingredients for pharmaceutical or cosmetic use.

US Patent application publication number 2007/0077281 teaches medical skin patches with a content of essential oils for treating colds and processes for their production. These medical skin patches are designed for treating colds by releasing essential oils through evaporation. They comprise at least one essential oil, at least one hydrophile polymer, at least one substance having an adsorbent effect or/and an emulsifier and at least one pressure sensitive adhesive polymer. The water content of the matrix is less than 5% by weight or even less than 1% by weight.

Nowhere in the art is it shown that hydrophilic polymer patches can actually provide transdermal delivery of essential oils. Nowhere in the art is it suggested that long lasting transdermal delivery of essential oils is effectively provided by hydrophilic polymer patches even in the absence of synthetic adhesive polymers.

### SUMMARY OF THE INVENTION

The present invention provides dermal patches that are advantageous in that they are substantially devoid of synthetic adhesive polymers. Thus the matrix of the dermal patches of the invention is a hydrophilic hydrocolloid that is derived from a natural exudate that is selected to be consistent with long term use without inducing, or inducing minimal irritation or discomfort in a human subject. These objectives are achieved with a patch comprising only natural polymeric ingredients or consisting essentially of natural polymeric ingredients. Synthetic additives are to be avoided generally or will be included only as minor components of the patch matrix. It is disclosed herein that these patches are simple to produce and to use and are capable of providing safe, comfortable and effective transdermal delivery of essential oils.

According to one aspect the adhesive dermal patches of the invention are formed from at least one hydrophilic polymer derived from a natural exudate, are substantially devoid of any pressure sensitive synthetic adhesive or any synthetic adhesive layer and contain above 5% (w/w) water. According to some embodiments the water content will be above 10% (w/w) water in the final product. In various embodiments, the final product may contain between 5 and 25% water, alternatively the water content will be in the range of 10-25%. According to some embodiments patches are not dried but some natural minimal evaporation might occur during processing. It is believed that this water content is beneficial to obtain the self adhesive dermal patches that do not require added synthetic pressure sensitive adhesives. Advantageously this water content diminishes the sensitivity to humidity in the environment or on the skin of the subject. This water content may also enable the dissolution of water soluble drugs and their inclusion/entrapment within the patch. According to some embodiments the patches of the invention may further comprise a therapeutic or cosmetic active agent. According to one embodiment the patches are sufficiently adhesive to readily stick to the skin of a subject and will not be sensitive to skin moisture.

According to some embodiments of the invention the patches of the invention are readily adhesive but are also sufficiently cohesive to be easily removed without leaving any significant residue or ideally no residue at all on the skin of the subject.

According to some embodiments the patches can be used for multiple applications. In some embodiments the patches of the present invention maintain their adhesiveness for prolonged periods and may be attached to the skin of a subject for a period of days without adverse effects. In other words according to some embodiments applying the patch to the skin of the subject and subsequently removing the patch does not decrease its adhesive properties. This is attributed to the fact that the patches of the invention are essentially devoid of pressure sensitive synthetic adhesives and the matrix is essentially a carrier that is also a uniformly adhesive polymeric matrix. Thus, the patch of the present invention is a single all inclusive layer that will serve as a carrier matrix and/or a reservoir for an active agent and as an adhesive. The dermal patch of the invention can be used as an all in one “drug-in adhesive”.

Patches can be produced from various natural exudates that support the desired properties of the patch matrix. Examples of suitable natural exudates include Sterculia foetida, Bauhinia variegata, Buchnania lanzan, Terminalia crenulata, Terminalia catappa, Terminalia belerica and gum karaya. According to certain exemplary embodiments the natural exudate is gum karaya.

The at least one natural polysaccharide exudate is typically dispersed within a non-solvent, also referred to herein interchangeably as a co-solvent. As used herein, a non-solvent is a liquid in which the polysaccharide is non-soluble and is able to disperse. In some embodiments, propylene glycol is used as a non-solvent for efficiently dispersing a powder of natural exudate, for example, gum karaya.

In some embodiments, the composition comprises about 20% to about 40% (w/w) non-solvent, also known as a co-solvent for suspension or dispersion of the polysaccharide. In some embodiments the composition comprises about 25% to about 35% (w/w) non-solvent.

The attributes required of the hydrophile polymer or exudate may be summarized as follows: 1) non-toxic; 2) minimal irritation; 3) good adhesive properties; 4) flexible and thereby able to conform to the curvature of the skin; 5) cohesive and does not leave appreciable residue; 6) economic to prepare; and 7) permits or promotes skin penetration of an essential oil contained therein.

Optionally, and advantageously the matrix may further comprise at least one additive selected from emulsifiers or surfactants, solvents or co-solvent solubilizers, dispersing agents and penetration enhancers. It is to be stressed that the use of synthetic additives will preferably be minimized. For example when used a synthetic surfactant may be used but such synthetic additives will preferably be no more than a single percent or a couple of percent of the end product. Additionally and optionally inert excipients may be added that serve as fillers, thickeners and the like. The fillers can be starches or other natural polysaccharides such as microcrystalline celluloses that are useful to modify the physical properties of the patches. For example, fillers can be used to maintain the integrity of the patch upon peeling away from the skin. In some embodiments they can be used to decrease the adhesiveness of exudates that are excessively adhesive. In some embodiments, the patch comprises about 5-20% filler, about 5-15%, about 5-10% filler.

The matrix may include additives useful for modifying (for example, increasing) the viscosity of the formulation, including but not limited to glycerol and propylene glycol. The use of such additives may also contribute to better absorbency of water and/or skin moisture.

According to another aspect the dermal patch of the invention comprises at least one hydrophile polymer, at least one essential oil, one surfactant or emulsifier, and is substantially devoid of any synthetic pressure sensitive adhesive. According to some embodiments the essential oil or mixture of essential oils comprises 1-10% (w/w) of the final product. According to some embodiments the essential oil or mixture of essential oils comprises 2.5-10% (w/w) of the final product. According to some embodiments the dermal patch has a water content above 5% (w/w) of the final product. According to some embodiments the dermal patch has a water content above 10% (w/w) of the final product.

The patch of the invention can be designed or may be cut to be in any suitable size of shape and readily conforms to the contours of the skin. In addition, thickness of the patch can be controlled. In general, the patch can be designed to be of any desired thickness. The thickness will be a function of the volume of the patch mixture and the area of the surface area. In general, the thickness of the patch ranges from tens of microns to a few millimeters, for example from about 20 microns to 5 mm, from about 50 microns to 3 mm, from about 50 microns to 5 mm. The skilled artisan will readily appreciate that for some embodiments, the preferred patch thickness is in the range of tens of microns while for others the patch thickness may be hundreds of microns or in the range of 1 to 5 mm, or 2 to 5 mm, or even 3 to 5 mm. In some applications it is desirable to provide a patch covering an area as small as possible. In other situations it may be desirable to cover a larger area in order to treat the maximal area.

The patch will have an internal side which will be attached the skin and an external surface facing outwards from the skin. According to some embodiments the patch will be supplied with a backing layer or liner covering the side of the patch that is intended for contact with the skin, which is removable prior to application to the skin of a subject.

According to additional embodiments the patch will be supplied with a cover sheet or cover layer on the external surface facing outwards from the skin that prevents absorption of moisture and contaminants from the environment. According to some embodiments the sheet or cover is a plastic cover which is removable prior to or after application of the patch to the skin. According to some embodiments the plastic cover may be maintained on the patch while it is worn by the subject.

According to alternative embodiments the cover layer is permeable to gas or water vapor to prevent an occlusive bandage effect.

According to another aspect the present invention provides methods for increasing the penetration of at least some of the components of an essential oil through the dermis of a subject. It is now disclosed as exemplified herein below that the patches of the present invention can be used to provide quantifiable transdermal penetration of essential oils. Thus, the patches of the present invention are useful in methods to promote transdermal penetration of at least some components of essential oils as compared to other known methods of applying essential oils to a subject.

Unexpectedly, it is now disclosed that the transdermal delivery afforded by the dermal patches of the present invention is effective over prolonged periods of time. The patches serve as a reservoir of the essential oils contained therein and achieve transdermal delivery over periods of many hours and even over the course of several days. They can be used continuously without any adverse effects or irritation to the skin. Importantly, they can even be used intermittently and thus applied, removed from and reapplied to the skin of a subject without losing adhesiveness or effectiveness.

These and additional aspects and features of the invention will become apparent in conjunction with the figures, the detailed description and the examples that follow.

### BRIEF DESCRIPTION OF THE FIGURES

FIG. 1. Typical stress-strain relationships. A) Patches containing no starch; B) Patches containing starch. EO=essential oil

FIG. 2. Stress values at 75% deformation for patches with or without (w/o) starch. Bars headed by different letters (a-d) within and between treatments indicate a statistically significant difference at p<0.05.

FIG. 3. Modulus of deformability at 30% deformation for patches with and without (w/o) starch. Bars headed by different letters (a-d) within and between treatments indicate a statistically significant difference at p<0.05.

FIG. 4. Typical compression-decompression relationships. A) Patches containing no starch; B) Patches containing starch. The patches were compressed to 20% deformation at a rate of 10 mm/min. EO=essential oil

FIG. 5. Percent recoverable work of patches with or without (w/o) starch. The patches were compressed to 20% deformation at a rate of 10 mm/min. Bars headed by different letters (a-e) within and between treatments indicate a statistically significant difference at p<0.05.

FIG. 6. Percent recoverable work of patches containing starch subjected to one compression-decompression cycle under deformations of 10% or 50% and compressed at a deformation rate of 10 mm/min Bars headed by different letters (a-c) within and between treatments indicate a statistically significant difference at p<0.05.

FIG. 7. Percent recoverable work of patches with or without (w/o) starch subjected to deformation rates of 0.1, 10, and 100 mm/min. The patches were compressed to 20% deformation. Different letters (a-d) within and between treatments indicate a statistically significant difference at p<0.05.

FIG. 8. Typical tack curve.

FIG. 9. Tackiness of patches with and without (w/o) starch. Bars headed by different letters (a, b) within and between treatments indicate a statistically significant difference at p<b 0.05.

FIG. 10. Typical peeling graph obtained by peeling a patch containing 2.5% essential oil and 10% starch from a skin model.

FIG. 11. Peeling force of patches with starch. Bars headed by different letters (a, b) within and between treatments indicate a statistically significant difference at p<0.05.

FIG. 12. Scanning electron micrograph of a patch without the inclusion of starch granules. Patch adhered to the skin model with no detectable space between them.

FIG. 13. Scanning electron micrograph of a patch with the inclusion of oval “bodies”: single or aggregated starch granules that are distributed in a homogeneous manner within the patch and are coated by its karaya gum matrix.

FIG. 14. Permeation profile of d-limonene for a dose of Valencia orange essential oil application through a patch to the rat's skin membranes.

FIG. 15. Accumulated concentration of linalool in blood samples after application of patches containing 7.5% lavender essential oil or direct smearing (massage-like simulation) of a mixture of almond oil and lavender oil.

FIG. 16. Decomposition of linalyl isovalerate in the blood.

FIG. 17. Accumulated concentration of camphor in blood samples after application of patches containing 7.5% lavender essential oil or direct smearing (massage-like simulation) of a mixture of almond oil and lavender oil.

FIG. 18. Accumulated concentration of linalool, linalyl acetate and camphor in blood samples after application of patches containing 7.5% lavender essential oil, as measured using GC-WAX column.

FIG. 19. Accumulated concentration of linalyl acetate, linalool, camphor, borneol and α-terpineol in blood samples after application of patches containing 7.5% lavender essential oil, as measured using GC-HP-5 column.

FIG. 20. Concentrations of different constituents extracted from the skin after application of patches containing 7.5% lavender essential oil.

FIG. 21. Proposed model for the mechanism underlying the delivery of essential-oil components through the skin.

### DETAILED DESCRIPTION OF SOME EMBODIMENTS OF THE INVENTION

The present invention relates to dermal patches comprising a bioadhesive composition comprising at least one polysaccharide exudate. As used herein, the term “bioadhesive’” refers to compositions that adhere to a surface such as skin without the need for additional wetting or hydration prior to use on the subject. The dermal patches of the invention contain sufficient water to achieve the desired adhesive properties upon contact with the skin of a subject. Typically, the patches will adhere to the skin within a second or two with minimal pressure.

The dermal patches of the present invention comprise a bioadhesive composition which is substantially devoid of synthetic pressure sensitive adhesives. As used herein, the term “substantially devoid” refers to less than 1%, preferably less than 0.1%, less than 0.01% (w/w).

In particular the present invention provides dermal patches that are suitable for transdermal or intradermal delivery of essential oils.

Essential oils are useful in many applications both in the field of cosmetics and in the field of pharmaceuticals. It is an object of the present invention to provide compositions comprising exclusively or at least consisting essentially of natural ingredients for the safe and effective delivery of essential oils to the skin of a subject.

Advantageously, as disclosed herein the major components of the dermal patch matrix will be effective in adhering to the skin of a subject while being non-toxic, non-irritating, economic, usable over prolonged periods of several days and potentially even re-usable for multiple applications to a subject. In addition, the dermal patch of the present invention may absorb a small amount of perspiration without losing its adhesive properties.

The present invention establishes the physical properties of hydrophilic hydrocolloid based dermal patches that enable the effective transdermal delivery of essential oils.

The essential oils are incorporated in a hydrophile, self-adhesive matrix which serves as a reservoir for these essential oils. According to some embodiments the dermal patch is supplied with a removable internal backing layer or liner on the side intended for adhesion to the skin. Typically the patch further comprises an external cover layer which, in the state of having been applied to the skin, may remain or may be removed. According to some embodiments the cover layer is moisture resistant. According to alternative embodiments the cover layer may be gas- and water vapor-permeable to prevent occlusion of the skin. Due to their hydrophile character, these patches are well tolerated by the skin, and an occlusion effect is prevented. Cover or sheet materials, including wovens (e.g. of polyester) or textile substances that exhibit the desired permeability properties may be used as the gas- and water vapor-permeable backing layer. Examples of suitable materials include open cell foamed plastics (e.g. polyurethane foam, polyethylene foam, plastic films rendered permeable by mechanical treatment, e.g. perforated polyethylene, polyethylene terephthalate and PVC films).

In additional embodiments, the hydrophile matrix, following its production and during storage, is covered on its intended skin-contact side with a detachable protective film. Suitable for this purpose are, for example, polyester or other plastics tolerated by the skin, such as polyvinyl chloride, ethylene vinyl acetate, vinyl acetate, polyethylene, polypropylene and cellulose derivatives, these films being made detachable by suitable surface treatment, such as siliconization. The skin patches of the present invention are preferably sealed in gas- and water vapor-tight packages.

Chemical Composition

The dermal patches according to the present invention contain at least one hydrophile polymer and having a water content above 5% by weight or even higher, during the manufacture as well as in the final product. The hydrophile polymers in the matrix, forming the basis of the formulations according to the present invention are capable of absorbing large amounts of moisture or water during the period in which they are applied on the skin, without losing their structural integrity and adhesiveness. The use of fillers, co-solvents (also known as non-solvents) and other additives may also contribute to better absorbency of water/skin moisture. Perspiration may be absorbed by the patch, resulting in moderate swelling of the patch.

The proportion of the hydrophile polymer(s) is preferably in the range of from 10 to 40% by weight, especially preferably in the range of from 20-30% by weight, relative to the total weight of said matrix.

Patches can be produced from different natural exudates. Examples of natural exudates include Sterculia foetida, Bauhinia variegata, Buchnania lanzan, Terminalia crenulata, Terminalia catappa, Terminalia belerica and gum karaya. Suitable hydrophile polymers are in principle all those natural hydrophile polymers that possess good swelling properties and are compatible with essential oils and well tolerated by the skin.

The at least one natural polysaccharide exudate is typically dispersed within a non-solvent. Non-limiting examples of suitable non-solvents include propylene glycol, dipropylene glycol, polyethylene glycol, butylene glycol, hexylene glycol, polyoxyethylene glycol, polypropylene glycol and ethylene glycol. In certain embodiments the non-solvent of the is propylene glycol.

In some embodiments, the composition comprises about 20% to about 50% (w/w) non-solvent. In some embodiments the composition comprises about 25% to about 35% (w/w) non-solvent.

The dermal patches according to the invention also contain at least one substance having a surfactant effect and/or at least one substance having an emulsifying effect. As was reported previously (US 2007/0077281), it has been found that by adding this type of substance it is possible, on the one hand, to prolong the time interval during which the matrix preparation containing the essential oils remains processable and, on the other hand, to prevent the occurrence of phase separation between the hydrophile matrix polymer(s) and the essential oil phase.

Suitable substances having an emulsifying effect are, in particular, the following substances and groups of substances, either individually or in combination: sodium palmitate, sodium stearate, triethanolamine stearate, sodium lauryl sulfate, gum Arabic, alkonium bromide, benzalkonium bromide, cetylpyridium chloride, cetyl alcohol, stearyl alcohol, higher branched fatty alcohols, partial fatty acids of polyhydric alcohols, partial fatty acid esters of sorbitan, partial fatty acid esters of polyoxyethylene sorbitan, sorbitol ether of polyoxyethylene, fatty acid esters of polyoxyethylene, fatty alcohol ethers of polyoxyethylene, fatty acid esters of saccharose, fatty acid esters of polyglycerol, lecithin and complex emulsifiers such as, for example, complex-emulsifying cetyl stearyl alcohol. In addition, other emulsifiers known to those skilled in the art may be utilized. The desired content of the emulsifiers should preferably not exceed one to several percent of the total weight of the matrix.

The hydrophile matrix of the skin patches according to the present invention exhibits pressure-sensitive adhesive properties on its own without the need for any synthetic pressure-sensitive adhesive polymer or combinations of such polymers.

The hydrophile matrix may further include viscosity modifiers, such as glycerol and propylene glycol. The use of such additives may also contribute to better absorbency of water and/or skin moisture. These additives may be present in the composition at a concentration of about 10% to about 40% (w/w), of about 20% to about 30% (w/w). In some exemplary embodiments, the composition comprises about 20% to about 30% (w/w) glycerol.

The hydrophile matrix containing the essential oils may in addition contain further formulation adjuvants, preferably moisturizers (e.g. anhydrous glycerol, propylene glycol or other polyhydric alcohols) or antifoaming agents. The proportion of the adjuvants may amount to 1 to 50% by weight, especially 5 to 30% by weight.

Suitable essential oils that can be used for the purpose of the present invention include, but are not limited to, lavender oil, orange oil, eucalyptol (cineol), menthol, thymol, borneol, bisabolol, mint oil, peppermint oil, spearmint oil, eucalyptus oil, camphor, turpentine oil, pine-needle oil, anise oil, fennel oil, thyme oil, rosemary oil, camomile oil, sandalwood oil, Davana oil and clove oil. Combinations of the aforementioned substances or mixtures of substances are also suitable.

In some exemplary embodiments, the essential oil is selected from the group consisting of lavender oil, orange oil.

Additional examples of suitable essential oils include Agar oil, Ajwain oil, Angelica root oil, Anise oil, Asafoetida, Balsam oil, Basil oil, Bergamot oil, Black Pepper essential oil, Buchu oil, Birch, Cannabis flower essential oil, Caraway oil, Cardamom seed oil, Carrot seed oil, Cedarwood oil, Chamomile oil, Calamus Root, Cinnamon oil, Citronella oil, Costmary oil, Costus Root, Cranberry seed oil, Cubeb, Cumin oil/Black seed oil, Cypress, Cypriol, Curry leaf, Davana oil, Dill oil, Elecampane, Fennel seed oil, Fenugreek oil, Frankincense oil, Galangal, Galbanum, Geranium oil, Ginger oil, Goldenrod, Grapefruit oil, Henna oil, Helichrysum, Horseradish oil, Hyssop, Idaho Tansy, Jasmine oil, Juniper berry oil, Laurus nobilis, Ledum, Lemon oil, Lemongrass, Lime, Litsea cubeba oil, Mandarin, Marjoram, Melaleuca See Tea tree oil, Melissa oil (Lemon balm), Mentha arvensis oil/Mint oil, Mountain Savory, Mugwort oil, Mustard oil, Myrrh oil, Myrtle, Neem Tree oil, Neroli, Nutmeg, Limonene, Oregano oil, Orris oil, Palo Santo, Parsley oil, Patchouli oil, Perilla essential oil, Pennyroyal oil, Peppermint oil, Petitgrain, Pine oil, Ravensara, Red Cedar, Roman Chamomile, Rose oil, Rosehip oil, Rosemary oil, Rosewood oil, Sage oil, Sandalwood oil, Sassafras oil, Savory oil, Schisandra oil, Spearmint oil, Spikenard, Spruce, Star anise oil, Tangerine, Tarragon oil, Tea tree oil, Thyme oil, Tsuga, Turmeric, Valerian, Vetiver oil (khus oil), Western red cedar, Wintergreen, Yarrow oil, Ylang-ylang and Zedoary.

The dermal patches may also include a penetration enhancer. Suitable penetration enhancers for transdermal application include, for example, alcohol and polyols.

In some embodiments, the dermal patch further comprises an active agent selected from a therapeutic agent and a cosmetic agent. Therapeutic and cosmetic agents useful in connection with the patch of the present invention include compounds or chemicals that are capable of dermal or transdermal administration.

Examples of therapeutic agents include anti-microbial agents including antibiotics, antifungal and antiviral agents; bacteriostatic agents; analgesics and analgesic combinations; anesthetic agents; anorexic agents; antiarthritic agents; antiasthmatic agents; anticonvulsants; antidiabetic agents; antiemetic and antidiarrheal agents; antihistamines; anti-inflammatory (steroidal and non-steroidal) and antipruritic agents; antimigraine preparations; antineoplastics; psychotherapeutics; antipyretics; antispasmodics; antiarrhythmics; antihypertensives; opioid antagonists; hormones; as well as pharmaceutically acceptable salts and esters thereof. The amount of therapeutic agent that constitutes a therapeutically effective amount can be readily determined by those skilled in the art with due consideration of the particular agent, the particular carrier, and the desired therapeutic effect.

Examples of cosmetic agents include anti-acne and anti-sebum agents, anti-oxidants, anti-aging, anti-scar and scar-, wrinkle- and pigment-reducing agents and moisturizers.

The composition of the present invention may further comprise one or more additives useful in the preparation or application of topically applied substances. For example, solvents, including alcohol, may be used to solubilize certain active agents. For pharmaceutically active agents having a low rate of permeation through the skin, it may be desirable to include a further permeation enhancer in the composition. Enhancers should be chosen to minimize the possibility of skin irritation, damage, and skin and systemic toxicity. Examples of suitable enhancers include, in a non-limiting manner, ethers such as diethylene glycol monoethyl ether (Transcutol®); surfactants such as sodium laurate, sodium lauryl sulfate (SLS), cetyltrimethylammonium bromide (CTAB), Poloxamer (231, 182, 184), Tween (20, 40, 60, 80) and lecithin; alcohols such as ethanol, propanol, octanol, benzyl alcohol, and the like; polyethylene glycol (PEG) and esters thereof; amides and other nitrogenous compounds such as benzalkonium chloride, urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids. The permeation/penetration enhancers may in some instances provide more than one benefit or operate via more than one mode of action. For example, benzalkonium chloride may be used as a preservative.

One example of a suitable preservative is about 0.2% quaternium-15 by weight of the mixture, but may also be paraben or other preservatives in small amounts generally less than 1% of the weight of the mixture.

The classification of agents used herein is made for the sake of convenience only and is not intended to limit any component to that particular application or applications listed.

Physical Properties

In exemplary embodiments the essential oils were included at concentrations of 2.5 to 10% within patches manufactured from gum exudate, propylene glycol, glycerol, emulsifier and water. The patches were mixed and formed at room temperature, and then tested for their mechanical properties with an Instron Universal Testing Machine. Relative to patches with no oil, oil inclusion (at 10% w/w) caused a reduction in patch strength from about 50 to 25 kPa and in degree of elasticity from about 73 to 63%. The same tendency was observed for other oil concentrations. Stiffness was not influenced at all. The roughness, gloss, structure and adhesiveness of the patches were also studied by profile-meter, glossmeter and scanning electron microscope. In summary, although inclusion of essential oil reduced the mechanical properties of the patches, a high proportion of essential oil can be included without adversely affecting patch integrity or eliminating their adhesiveness to the skin.

In some embodiments, the degree of elasticity of the patch is at least 55%, at least 60%.

The adhesiveness of the dermal patch can be quantified by a novel design of the conventional probe-tack tester, specifically adapted for use with tacky hydrogels. A full description of the apparatus is given, for example, in Ben-Zion et al. (2008). An exemplary procedure is described below. The conventional test method is detailed in the American Society for Testing Materials, Designation D-2979-01, under the jurisdiction of ASTM Committee D-14.50 on adhesives.

In some embodiments, the maximal tack force required to separate the patch from a skin model is in the range of 0.5 to 4 N, or in the range of 0.5 to 2.5 N. In some embodiments exemplified herein the tack force was in the range of 0.5 to 2.1 N.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

In examples 1-4 below, physical properties of patches based on gum karaya exudates which contain different concentrations of the essential oil Lavandula angustifolia were tested.

The patches were prepared by mixing a first phase composed of distilled water, glycerol, the essential oil, Tween 80 (as an emulsifier) and optionally potato starch as a filler, with a second phase composed of karaya gum powder and propylene glycol (used to suspend the karaya gum powder).

The final composition of these exemplary patches is as follows:

| | |
|---|---|
| Distilled water | 13.6 to 23.6% (w/w) |
| Glycerol | 21.1% (w/w) |
| (Sigma Chemical Co., St. Louis, MO) | |
| (Optional) Potato starch (Merck, Darmstadt, | 10% (w/w) |
| Germany) | |
| Propylene glycol (Merck) | 27.7% (w/w) |
| Karaya gum powder (bark-free, HPS-grade | 20.0% (w/w) |
| (hand-picked selected, summer crop, 200 μm) | |
| (Sigma) | |
| Lavandula angustifolia essential oil | 2.5 to 10% (w/w) |
| (“Light of the Desert”, kibbutz Urim, Israel) | |
| Tween 80 (Sigma) | 1% (w/w) |

Gum purity was verified by analysis of its infrared spectrum which proved to be characteristic with respect to many commercial samples from various sources. The two phases were prepared separately, stirred for 5 min at ambient temperature and kept at −20° C. for half an hour in order to slow the gelation reaction, which is otherwise immediate. The patches were then mixed together and quickly poured into a small Petri dish (height 5 mm, diameter 40 mm) or into a rectangular mold with dimensions of 11×10×0.5 cm (length×width×thickness) to form the final patch upon solidification. All patch types were prepared in two separate batches.

The pH of the patches was determined by pH meter (Model C830, Consort, Belgium) and pH electrode (Model 8163BN, Thermo, Orion, UK). Three replicates were carried out per sample. The skin has a pH of 4 to 6; consequently, if the pH of the patch lies outside that range, it could potentially irritate the skin. The pH of karaya-essential oil patches ranged from 4.44 to 4.65±0.007, thus falling within the required pH range.

Statistical analyses described in the examples below were conducted using JMP software (SAS Institute 2007, Cary, N.C.), including ANOVA and Tukey-Kramer Honestly Significant Difference test for comparisons of means, with p≦0.05 considered significant.

### Example

### Compression Tests

The mechanical properties of a patch are important since patches are designed to be compressed against the skin in order to achieve suitable contact followed by adhesion. The compression tests were performed using a universal testing machine (UTM; Instron model 5544, Instron Corporation, Canton, Mass.). Cylindrical samples with dimensions of 8×5 mm (diameter×height) were uniaxially compressed to about 90% between flat plates at a deformation rate of 10 mm/min to study their stress-strain relationships. Average stresses at 25%, 50%, and 75% strain were calculated. The UTM was connected to a computer by an analog-to-digital conversion interface card. The crosshead movements were controlled through the computer with “Merlin” software, supported by Instron. The UTM collects data as volts vs. time and then converts them to stress vs. strain. The corrected stress, σ(t), was calculated as follows:

σ(t)=[F(t)(H₀−ΔH(t))]/A₀H₀

where H₀ is the initial specimen length, ΔH(t) is the absolute deformation, F(t) is the force at time t and A₀ is the crosssectional area of the original specimen.

The engineering strain ε_{E} was calculated as:

$${ɛ}_{ɛ} = \frac{Δ H}{{H}_{0}}$$

where ΔH is the total deformation divided by the initial specimen length. All reported results are means of four to eight replicates.

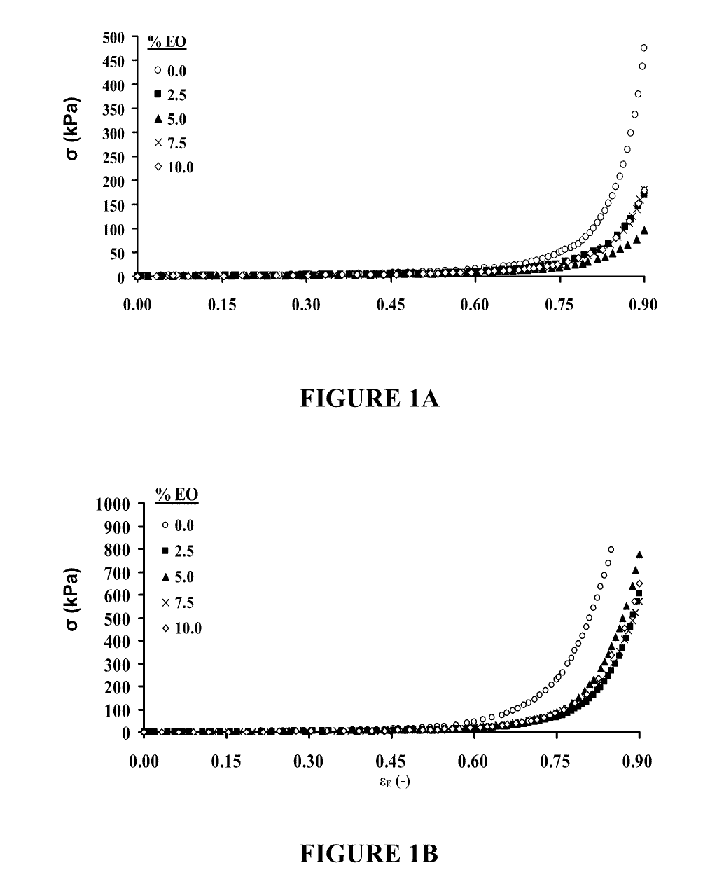

Patches with or without the potato starch filler were compressed up to about 90% deformation, and typical stress-strain relationships are demonstrated in FIG. 1. No visible signs of failure were observed during or after completion of the compression.

Stress at strain values of 25%, 50% and 75% for patches with or without starch filler are summarized in Tables 2 and 1 hereinbelow, respectively.

TABLE 1
Stress at different strain values for gum
karaya-essential oil patches (no filler)

| | Stress at 25% strain | Stress at 50% strain | Stress at 75% strain |
|---|---|---|---|
| % EO | (kPa) | (kPa) | (kPa) |
| 0 | 2.5^{a} ± 0.4 | 8.5^{a} ± 0.4 | 50.3^{a} ± 4.6 |
| 2.5 | 2.1^{a} ± 0.6 | 6.1^{b} ± 0.5 | 25.0^{b} ± 5.3 |
| 5 | 1.9^{a} ± 0.1 | 5.7^{b} ± 0.2 | 19.8^{b} ± 1.1 |
| 7.5 | 2.0^{a} ± 0.3 | 5.5^{b} ± 0.3 | 23.3^{b} ± 3.5 |
| 10 | 1.9^{a} ± 0.3 | 5.5^{b} ± 0.4 | 25.6^{b} ± 3.3 |

Results are expressed as mean ± standard error. Different superscript letters (a, b) within a column indicate a statistically significant difference at p < 0.05.

TABLE 2
Stress at different strain values for gum
karaya-essential oil patches (with filler)

| | Stress at 25% strain | Stress at 50% strain | Stress at 75% strain |
|---|---|---|---|
| % EO | (kPa) | (kPa) | (kPa) |
| 0 | 3.4^{a} ± 0.1 | 17.3^{a} ± 0.8 | 225.6^{a} ± 18.97 |
| 2.5 | 2.2^{ab} ± 0.1 | 9.3^{b} ± 0.1 | 66.7^{b} ± 3.2 |
| 5 | 2.7^{ab} ± 0.3 | 10.6^{b} ± 1.8 | 70^{b} ± 10.1 |
| 7.5 | 2.2^{b} ± 0.2 | 8.5^{b} ± 0.9 | 70.5^{b} ± 13.6 |
| 10 | 2.1^{b} ± 0.4 | 8.1^{b} ± 1.3 | 68.6^{b} ± 21.8 |

Potato starch at 10% was used as filler. Results are expressed as mean ± standard error. Different superscript letters (a, b) within a column indicate a statistically significant difference at p < 0.05.

As can be seen from FIG. 1A and Table 1, differences in the mechanical properties of patches containing various amounts of essential oil are better detected at the initial point of the curves' separation, i.e., at about 25% to 30% deformation; from that point on the observed stress at a particular strain value differs. At a strain of 25% with inclusion of essential oil at 2.5% to 10.0%, there was some decrease in the stress values in parallel to the increase in essential oil, although this decrease was not significant. However, at higher strains, i.e., 50% and 75%, with inclusion of different percentages of essential oil in the patches, a significant difference in stress values was observed, in particular between patches with no essential oil and those including essential oil. In other words, at 50% and 75% strain, inclusion of 2.5% essential oil was sufficient to generate a significant difference (decrease) in the stress values.

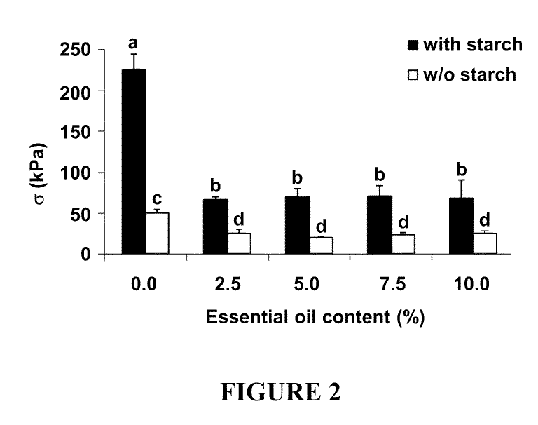

As can be seen from FIG. 1B and Table 2, patches that contained a starch filler appeared to present higher stresses at a given strain in comparison to those patches that did not include filler. When comparing data presented in Tables 1 and 2 (FIG. 2), it is apparent that the addition of starch strengthened and stabilized the patch. The patch matrix became denser (more tightly packed) due to the included starch granules and, as a result, more resistant to the applied stress. Patches with no essential oil appeared to be stronger than those that included any percentage of entrapped essential oil. In addition, all patches that included fillers were stronger than those that did not.

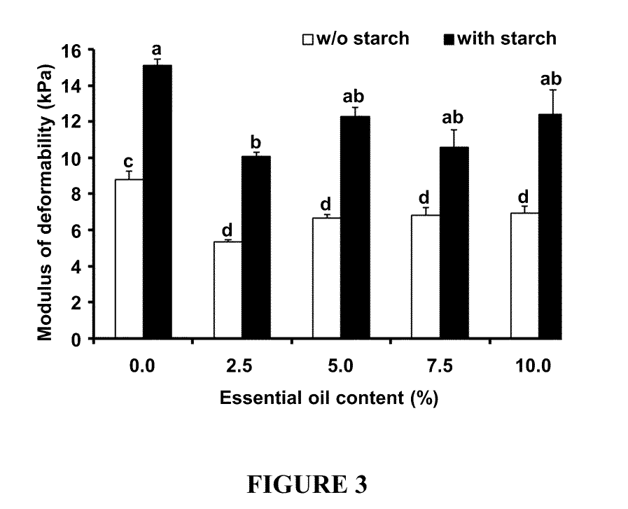

The modulus of deformability was calculated at 30% deformation where the relationship between stress and strain is highly linear (R²=0.97 or higher) (FIG. 3). Starch addition consistently increased the stiffness of the gum karaya patch and as a result, its resistance to deformation. The main change observed for both types of patches, with and without starch (FIG. 3), was the decrease in their deformability modulus upon inclusion of essential oil, even at its lowest amount (2.5%). In other words, the hydrocolloidal patch's texture is highly influenced by the inclusion of the main components of lavender essential oil, namely, linalool, and linalyl acetate.

### Example 2

### Elasticity Tests

To achieve attachment, a user presses the patch against the skin (tens of percentage points of deformation are involved) until it adheres to it. When pressure is removed the patch remains glued to the skin and attempts to recover its initial dimensions. This is approximately simulated by applying one cycle of compression-decompression to the patch during the degree of elasticity test, and results of this test may therefore be useful to both the manufacturer and the consumer.

The elasticity tests were performed as follows: cylindrical samples with dimensions of 8×5 mm (diameter×height) were subjected to compression-decompression cycles at predetermined deformations of 10%, 20%, or 50% using the UTM. Talc granules (MW: 379, particle size: <5 μm) were applied to both sides of the patch to prevent their adherence to the moving plate, which results in “negative” areas in the stress-strain curves. Crosshead speed was the same in both directions (0.1, 10, or 100 mm/min) The “degree of elasticity” has been defined as the ratio between recoverable and total compressive deformation, or as a percentage. It is calculated by the ratio between recoverable and total work, i.e.:

Degree of elasticity=Recoverable work (%)=(Recoverable work/Total work)×100

For the compression-decompression cycles, the areas under the stress-strain curves were calculated using the trapezoidal method as follows: n number of trapezoids were circumscribed under a curve, and then their areas were summed The area under the decompression curve was presented as percent of total work. All reported results are means of four to eight replicates taken from two separate batches.

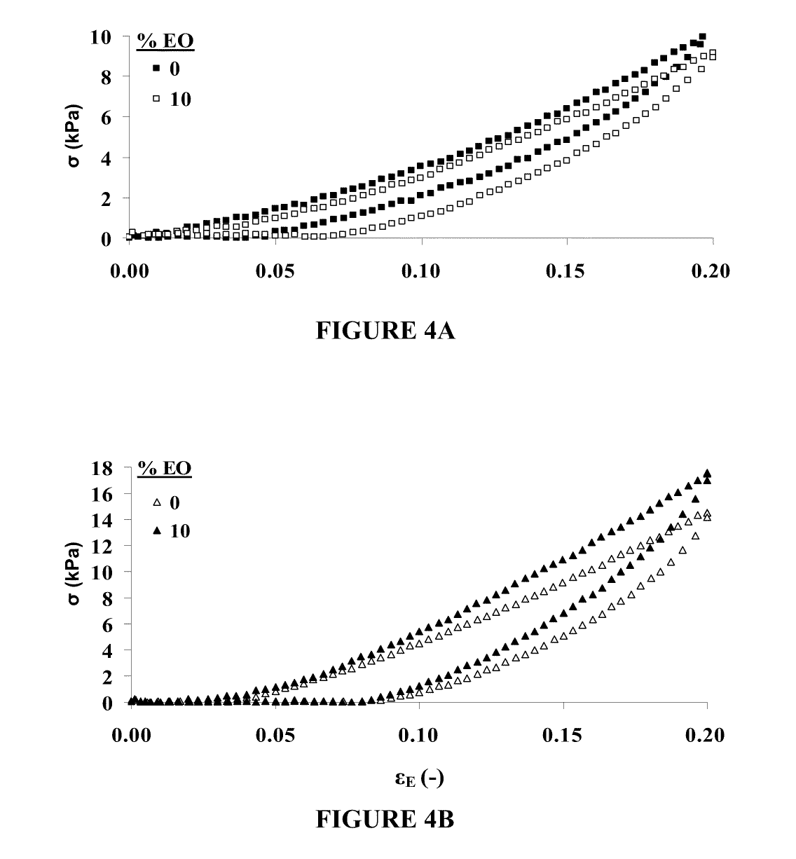

Typical compression-decompression relationships of patches including 0% or 10% essential oil with or without starch as a filler were studied and are demonstrated in FIGS. 4A+B.

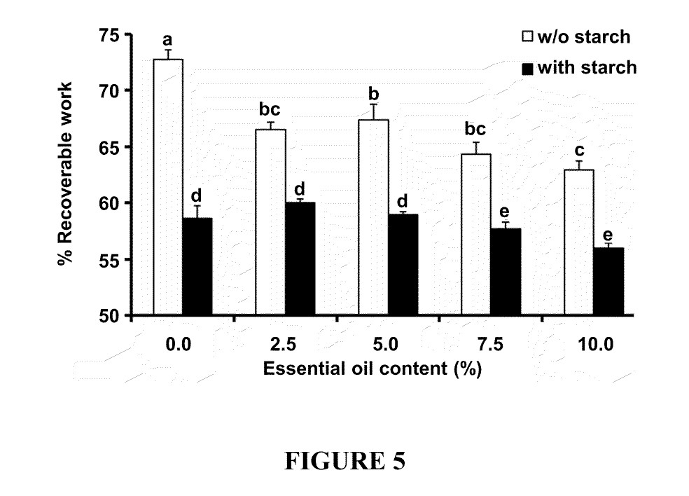

The influence of the included essential oil on the percent recoverable work of the patches with and without starch was calculated from the curves in FIG. 4 and is demonstrated in FIG. 5. Essential-oil inclusion caused a significant reduction in the patches' percent recoverable work (i.e., their degree of elasticity). For patches without essential oil, degree of elasticity values of 73.0±0.9% were observed, while for patches with included essential oil in the range of 2.5% to 10%, calculated degree of elasticity values fluctuated between 63.0%and 67.0%. The inclusion of starch in the patch decreased the recoverable work to 58.6±1.0%. Inclusion of 2.5% or 5.0% essential oil in the patch did not change its percent recoverable work significantly. However, the inclusion of 7.5% or 10.0% essential oil within the gum karaya-starch patches significantly reduced their percent recoverable work. Nevertheless, these findings for both patches, with and without starch, demonstrate that even after the inclusion of essential oil, the patch retains its high elastic properties and can be regarded as an elastic body.

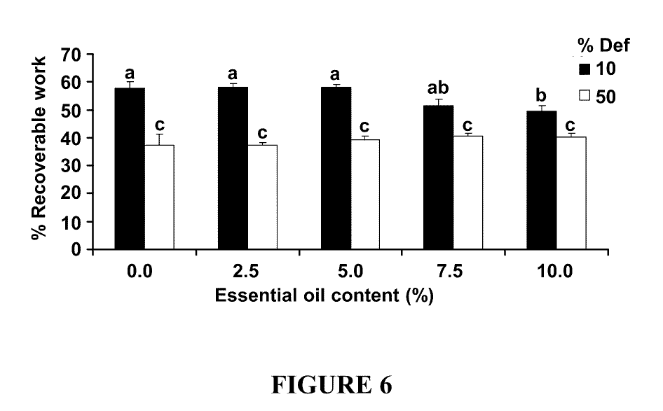

The deformation to which a patch is compressed is well known to influence its percent recoverable work. This hypothesis was checked by compressing the patches to 10 and 50% deformation (FIG. 6): percent recoverable work (i.e., the degree of elasticity) decreased as percent deformation increased. This was likely due to internal damage which probably occurred within the patch during the compression.

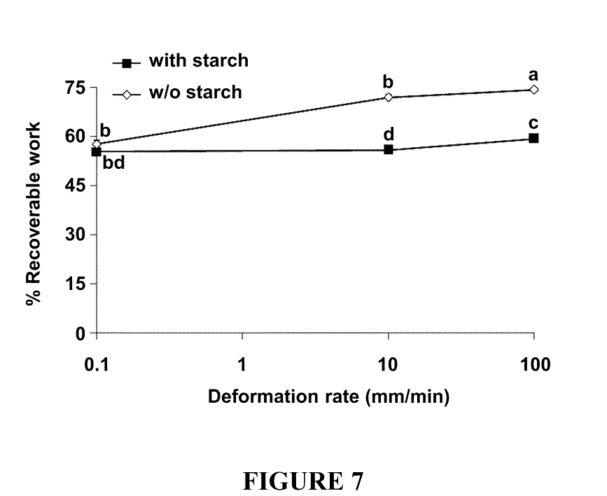

Deformation rate may also influence the elastic properties of the patches reflected by their percent recoverable work, and therefore patches were passed through one cycle of compression-decompression at deformation rates of 0.1, 10, and 100 mm/min, and then the average recoverable work under those conditions was calculated and compared (FIG. 7). For patches with and without inclusion of a filler (potato starch) there was no difference in percent recoverable work at a deformation rate of 0.1 mm/min However, a significant change was observed when the rate of deformation “jumped” to 10 and 100 mm/min It appears that at these latter rates, for both types of patches (with and without starch), the higher the deformation rate the bigger the recorded percent recoverable work. In addition, it appears that inclusion of starch granules reduced the calculated percent recoverable work. This kind of behavior could result from a reduction in the elastic properties of such patches.

### Example 3

### Probe-Tack Tests

Good adhesive properties are important for the patch's ability to efficiently serve as a reservoir for drugs for either topical or transdermal delivery.

Probe-tack tests were performed in order to evaluate adhesive properties of patches with and without starch, with different essential oil content. The tests were carried out using a novel design of the conventional probe-tack tester, specifically adapted for use with tacky hydrogels. A full description of the apparatus is given, for example, in Ben-Zion et al. (2008) J Adhes Sci Technol, 22:205-16. This specialized device is capable of detecting first contact between the probe and a pressure-sensitive adhesive (PSA) and of determining this contact as the initial dwell time. The probe test was performed in a custom-made apparatus connected to the UTM. The tip of a cleaned probe-20 mm diameter of adherend (skin model), is brought into contact with the adhesive (patch) at a controlled rate of 100 mm/min for 2 s. Then the bond formed between the skin and patch is detached at the same rate. Prior to the probe-tack test, the skin model was immersed in distilled water for 5 s to reach a relative humidity of about 25%, which is typical of the stratum corneum. Tack was measured as the maximum force required to separate the patch from the skin model. Three replicates were carried out per sample.

The skin model was prepared in accordance with U.S. Pat. No. 4,877,454 (Charkoudian et al.) to serve as a substrate in the probe-tack test. Porcine skin gelatin 225 bloom (7 g) (Sigma) was dissolved in 58.1 g of water at 50° C. with stirring. Then 0.035 g of propylparaben as a preservative (Sigma), 3.15 ml of sodium hydroxide solution (4%, w/w) (Frutarom, Haifa, Israel) and 0.35 g of glycerol were added. Ceraphyl GA (3 g) was added (Van-Dyk, Belleville, N.J.) as the lipid component in the skin model, resulting in a white emulsion. Before pouring the emulsion into a roughened mold, in accordance with Charkoudian's patent, 2.77 ml of formaldehyde solution (3%, w/w) was added. The mixture was allowed to set and dry under ambient conditions. After 24 h, the resultant skin model was carefully removed from the mold. The average thickness of the skin model was measured with a thickness meter. Roughness of the skin model was measured using a portable surface-roughness tester (Surftest-301, Mitutoyo Corp., Tokyo, Japan). Three measurements of Ra, recognized as representing an average roughness in practice, were made. Ra was calculated as:

$$Ra = \frac{1}{l} {\int }_{0}^{l} \vert y \vert d x$$

where l=evaluation length, and ∫|y|dz=total area of the peaks and valleys. Rz (average of the vertical distances from the highest peaks to the lowest valleys within five equal sampling lengths) was also determined, to characterize the aforementioned surfaces. The Ra and Rz measurements were taken in both the “x” and “y” dimensions of the plane surface of the skin model. Results are given as arithmetic mean±standard deviation (SD) for an evaluation length of 7.5 mm at a speed of 0.5 mm/s The average Ra values in the “x” and “y” dimensions were 19.51±0.15 and 19.61±0.66 μm, respectively. The average Rz values in the “x” and “y” dimensions were 98.6±8.7 and 94.6±3.8 μm, respectively. Ra values for both pig and human skin have been reported at 20±3 μm, similar to the Ra values obtained here for the skin model, indicating that the model mimics the topography of human skin and can serve as a good substitute for the study of patch adhesion.

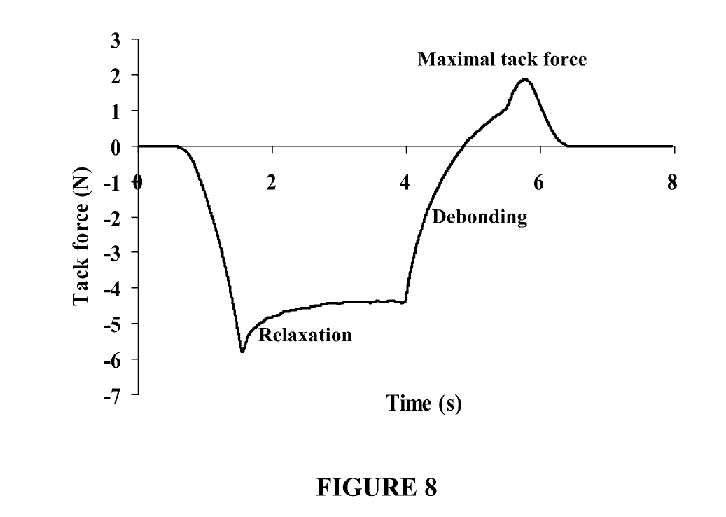

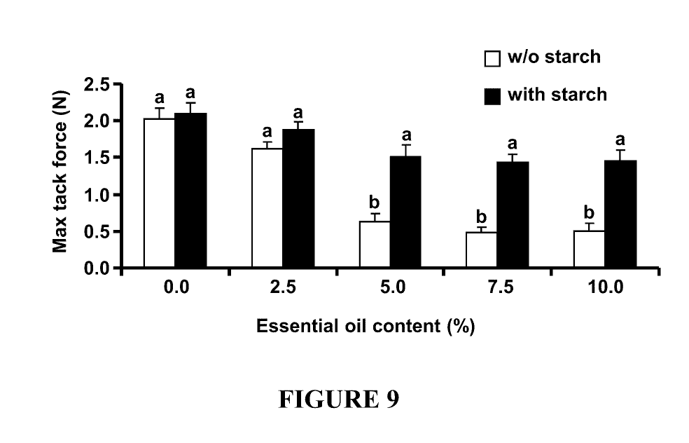

The adhesive properties of karaya-essential oil patches with and without starch were studied. A typical tack curve is demonstrated in FIG. 8, composed of the following components: first the patch is compressed; then the compression is stopped at a predetermined deformation, and force relaxation takes place followed by a debonding process, reaching a maximal tack force and then declining to zero tack force upon detachment. For the different patches, the maximal tack force required to separate the patch from a skin model was measured. FIG. 9 reveals that for patches without starch inclusion, the higher the inclusion of essential oil within the patch, the larger the decrease in its tackiness; maximum force values of 2.09±0.15 N were detected for patches without inclusion of essential oil whereas for patches with 10% included oil the maximum force values decreased to 0.49±0.11 N. Starch inclusion increased the maximum recorded tack force values. A significant difference between patches with and without starch inclusion was observed when the content of the entrapped essential oil was 5% or higher. The increase in maximum force values as a result of starch inclusion may be due to changes in the surface properties of the patch that is contacting the substrate, be it a skin model or skin. As previously stated, the inclusion of essential oil within the patch without starch reduced its maximum force value, since the included oil is not adhesive and it potentially reduces patch adhesiveness. Inclusion of starch within a patch that already includes essential oil creates a different situation, in which non-gelatinized, non-tacky, rigid starch granules replace some of the essential oil “regions” present on the surface of the patch; as a result, the patch's hydrophobicity decreases and its tackiness increases. This phenomenon is further emphasized at higher oil inclusions.

### Example 4

### Peeling Tests

The adhesion properties of the patches were also studied by 90° peeling test. Patches are regularly peeled when they need to be replaced. In addition, in parallel to designing a patch that can withstand water (washing), sweat, and so on, and stay on the body for as long as required by its destined use, it is beneficial to develop patches that can be peeled and re-adhered without losing their adhesion property.

The patches were peeled from a skin model sample as described, for example, in Portelli et al. (1986) In: Hartshorn S R, editor, Structural adhesives chemistry and technology, New York: Plenum Press; p. 407-49; and Ben-Zion et al. (1997) Food Hydrocolloids, 11:429-42. The skin model was immersed in distilled water for 5 s to reach a relative humidity of about 25%. The patch was attached to the skin model surface, and peeling tests were carried out with the UTM. During the test, a graph showing the peeling force (g force/cm) as a function of peeling length (cm) was obtained. Rectangular samples with dimensions of 11×3.3×0.5 cm (length×width×thickness) were used. Six replicates were carried out per sample.

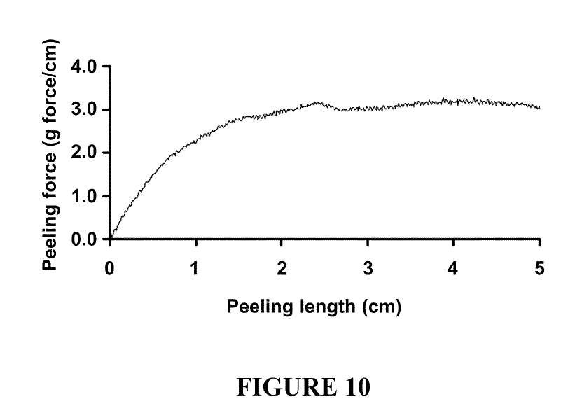

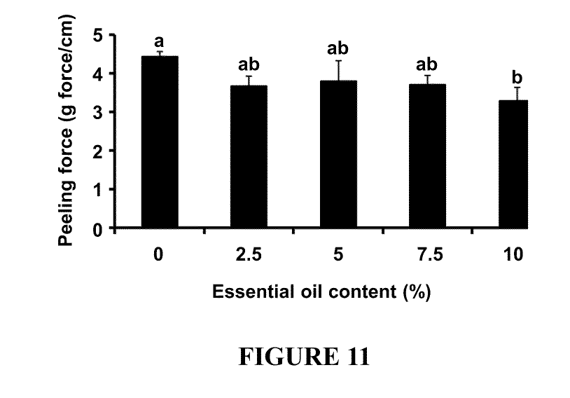

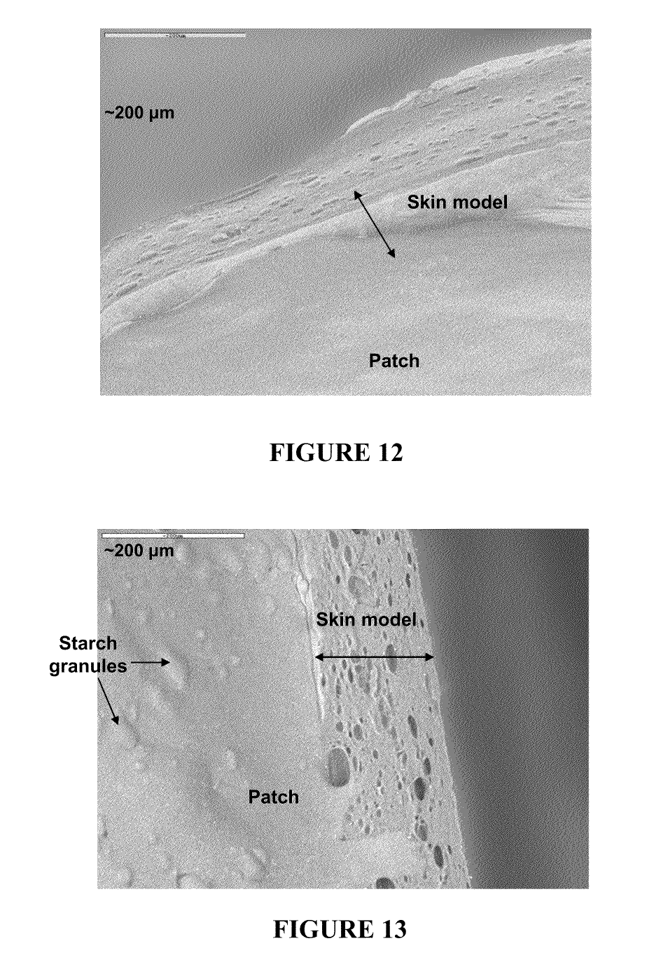

For test purposes, peeling from skin is simulated by attaching one side of the patch to a skin model, and attaching the other side to the grip of a UTM, at a 90° angle. The patch is then peeled at a deformation rate of 65 mm/min The peeling of karaya-essential oil patches from model skin was not possible due to stretching, and then tearing of the rectangular-shaped patches during the test. To overcome its capacity to overstretch and to stabilize the patch (see earlier), 10% potato starch was added to the patch formulation. The typical peel relationships for these fortified patches are demonstrated in FIG. 10. Essential oil inclusion caused a reduction in peeling force, but the difference was not statistically significant for patches with 2.5% to 7.5% included oil (FIG. 11). The only significant reduction in peeling force was observed for patches with 10% included essential oil resulting in 3.2±0.3 g force/cm, compared to patches without essential oil that had a mean peeling force of 4.4±0.1 g force/cm. These results were in agreement with those obtained in the probe-tack test: inclusion of essential oil in the patch reduced its adhesion to the skin model. SEM micrographs of patches without or with the inclusion of starch granules are shown in FIGS. 12 and 13, respectively. Both figures show that the patches, whether they include starch granules or not, adhered to the skin model with no detectable spaces between them. FIG. 13 also shows oval “bodies” with a diameter of about 50 μm which are not apparent in FIG. 12. It is hypothesized that these bodies are single or aggregated starch granules that are distributed in a homogeneous manner within the patch and are coated by its karaya gum matrix.

### Example 5

### Ex-Vivo Transfer of Essential-Oil Constituents Through the Skin

In Examples 5-6 below, the patches were prepared by mixing a first phase composed of distilled water, glycerol, either Lavandula angustifolia essential oil or Valencia orange oil, Tween 80 (as an emulsifier) and optionally potato starch as a filler, with a second phase composed of karaya gum powder and propylene glycol (used to suspend the karaya gum powder).

The composition of the final patches is as follows:

| | |
|---|---|
| Distilled water | 13.6 to 23.6% (w/w) |
| Glycerol | 21.1% (w/w) |
| (Sigma Chemical Co., St. Louis, MO) | |
| (Optional) Potato starch (Merck, Darmstadt, | 10% (w/w) |
| Germany) | |
| Propylene glycol (Merck) | 27.7% (w/w) |
| Karaya gum powder (bark-free, HPS-grade | 20.0% (w/w) |
| (hand-picked selected, summer crop, 200 μm) | |
| (Sigma) | |
| Lavandula angustifolia essential oil | 7.5% (w/w) |
| (“Light of the Desert”, Kibbutz Urim, Israel) or | |
| Valencia orange oil (Kibbutz Givat Haim, Israel) | |
| Tween 80 (Sigma) | 1% (w/w) |

Ex-vivo release studies were performed in Franz diffusion cells (FCs) (PermeGear, Hellertown, Pa.). Stomach skin pieces (1.2 cm×1.2 cm) from a male rat, which were stored in the freezer for 1 month, were thawed at room temperature and placed in glass FCs. The surface area for absorption was 0.64 cm2. The FCs were thermoregulated with a water jacket at 32° C. The 4.5-ml volume of the receptor chamber of the FC was filled with a receptor solution containing 5% (w/v) bovine serum albumin (BSA) diluted with phosphate buffer pH 7.4. Either 0.38 g of patch or almond oil (both containing 7.5% essential oil) were applied to the donor compartment. The receptor solution was continuously agitated with a magnetic stirrer. During the experiments, the donor compartments and sampling arms were sealed to prevent evaporation. The fluid in the receptor chamber was removed after different periods of time (0, 6, 12, 24, 36 and 48 h) and replaced with fresh phosphate buffer solution. Samples were stored at 4° C. after sampling, prior to analysis. Samples were analyzed in a gas chromatograph (Agilent Technologies, Santa Clara, Calif.) with a WAX 30 m×0.32 mm×0.25 μm (Agilent) column.

Transdermal delivery of essential-oil components through rat skin from both lavender essential oil, which includes a high proportion of terpenoids, and Valencia orange essential oil, which includes a considerable amount of terpene, entrapped within gum-karaya-based patches, was examined. In addition to the regular patch adhered to the skin to be tested for essential-oil delivery by the traditional FCs and by in vivo tests (detailed below), another experiment was performed in which the skin was smeared with an almond-essential oil mixture, in order to partially imitate the situation in which the skin is massaged in an aromatherapy session. All experiments were conducted at ambient temperature and the inclusion of the essential oil as part of the almond oil-essential oil mixture (for the massage-like simulation) was equal in quantity and proportion to the inclusion of essential oil within the patch. This additional experiment (i.e. massage simulation) was performed because an earlier report (Jager et al., 1992) on percutaneous absorption of lavender oil from massage oil had reported the possible transdermal delivery of essential-oil components into the bloodstream. The main components of lavender and orange essential oils are given in Table 3 hereinbelow.

TABLE 3
Composition of lavender and orange essential oils ^{z}

| Lavandula angustifolia essential oil | | Valencia orange essential oil | |
|---|---|---|---|
| Ingredient | % in oil | Ingredient | % in oil |
| Linalool | 36.18 | d-Limonene | 95.17 |
| Linalyl acetate | 32.31 | Myrcene | 1.86 |
| (E)-Caryophyllene ^{y} | 4.73 | α-Pinene | 0.42 |
| (E)-β-Farnesene ^{y} | 3.64 | Decanal | 0.28 |
| Borneol | 2.87 | Linalool | 0.25 |
| (Z)-β-Ocimene ^{y} | 1.61 | Sabinene | 0.12 |
| Caryophyllene oxide | 1.47 | β-Pinene | 0.12 |
| Hexyl butanoate | 1.28 | Geranial | 0.10 |
| Camphor | 1.17 | Neral | 0.07 |
| α-Santalene | 1.01 | Dodecanal | 0.07 |
| α-Terpineol | 0.99 | Citronellal | 0.05 |

^{z} Information was supported by the suppliers of the essential oil.
^{y} E stands for entgegen, i.e. opposite sides of a double bond; Z stands for zusammen, i.e. same side of a double bond.

In lavender oil, the two main ingredients are the terpenoids linalool and linalyl acetate, which together account for 68.4% of the composition of the oil. In the Valencia orange essential oil, the main ingredient is the terpene d-limonene. Although it was chosen to monitor the possible transfer of linalool, linalyl acetate and camphor (from lavender essential oil) and d-limonene (from Valencia orange essential oil) through the skin, it is important to note that an ingredient's higher proportion in the essential oil does not imply superior transfer abilities through the skin. In fact, penetration depends on many other factors, some of which were investigated in this study.

The accumulated concentrations of linalool, linalyl acetate and camphor that were transferred to the buffer from the patch through the skin in the ex-vivo experiments are reported in Table 4 hereinbelow.

TABLE 4
Accumulated concentrations of linalool, linalyl
acetate and camphor transferred from gum karaya-
Lavandula angustifolia essential oil patches^{z}

| Elapsed time | Linalool | Linalyl acetate | Camphor |
|---|---|---|---|
| (h) | (mg/L) | (mg/L) | (mg/L) |
| 6 | 16.49 ± 2.81^{a} | 0.15 ± 0.14^{a} | 0.51 ± 0.33^{a} |
| 12 | 34.46 ± 1.20^{b} | 0.26 ± 0.25^{a} | 0.74 ± 0.02^{b} |
| 24 | 61.64 ± 3.83^{c} | 0.37 ± 0.01^{a} | 1.53 ± 0.13^{c} |

^{z}At time zero, no traces of the three components were detected. Results are expressed as mean ± standard error.
^{a,b}Different superscript letters within a column indicate a statistically significant difference at P < 0.05.

At time 0, there was no transfer of any of the ingredients into the buffer. For the three tested ingredients at 6, 12 and 24 h from the start of the experiment, the more time allowed for the transfer, the higher the concentration of the diffused ingredient. Significant differences in concentration vs. time were only observed for linalool and camphor. For linalyl acetate, an increase in the amounts of the diffused ingredient was observed but the difference over time was not significant. It is important to note that the soft gum karaya patch, after its first compression to the skin, fits itself neatly to the skin's curvatures and thus the skin faces a homogeneous and isotropic distribution of essential oil droplets at a similar average distance from the skin. Table 5 hereinbelow presents the accumulated concentrations of linalool, linalyl acetate and camphor that were transferred into the buffer through the skin after the latter had been smeared with a mixture of almond-lavender essential oil.

TABLE 5
Accumulated concentrations of linalool, linalyl acetate,
camphor diffused from rubbing a mixture of Lavandula angustifolia
essential oil and almond oil on the skin^{z}

| Elapsed time | Linalool | Linalyl acetate | Camphor |
|---|---|---|---|
| (h) | (mg L⁻¹) | (mg L⁻¹) | (mg L⁻¹) |
| 6 | 31.07 ± 36.72^{a} | 2.30 ± 2.24^{a} | 0.01 ± 0.03^{a} |
| 12 | 48.34 ± 50.41^{a} | 3.33 ± 3.23^{a} | 0.89 ± 0.98^{a} |
| 24 | 94.78 ± 0.19^{a} | 5.62 ± 4.10^{a} | 1.78 ± 0.06^{a} |

^{z}Results are expressed as mean ± standard error.
^{a,b}Different superscript letters within a column indicate a statistically significant difference at P < 0.05.

Although an increase in the concentration of the ingredients versus elapsed time was observed, the differences in a particular component's concentration with time were not significant. Except for 6 h (camphor), the accumulated diffused amounts were found to be higher than those observed when the adhered patch was glued for the length of the experiment. Nevertheless, it is important to note that in the case of hand massaging, the coefficient of variance was very high, possibly due to uneven distribution on the skin.

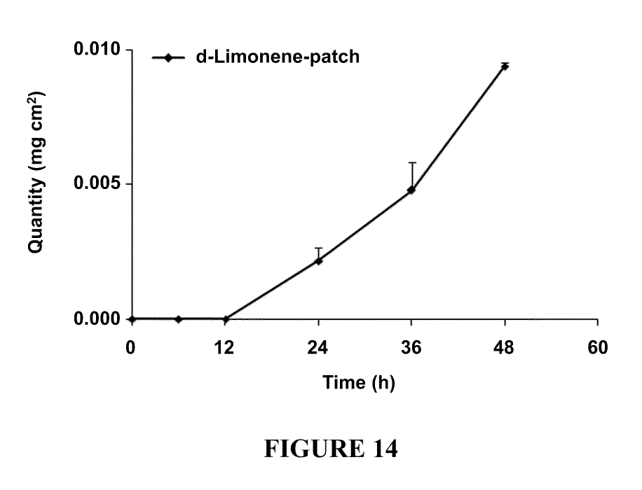

FIG. 14 presents a typical permeation profile for a dose of Valencia orange essential oil application through a patch to the rat's skin membranes. The steady-state flux was calculated from the slope of the linear portion of the curve of accumulated amount of essential oil per unit area vs. time. The calculated steady flux for d-limonene (in this case) was 1.9×10⁻⁴ mg.cm⁻² h⁻¹. The calculated steady-state fluxes for linalool and camphor transferred from a patch (curves not shown) were 0.018 and 4.3×10⁻⁴ mg.cm² h⁻¹ respectively, versus 0.027 and 5.7×10⁻⁴ mg.cm⁻² h⁻¹ when these ingredients were transferred through the skin by rubbing in a mixture with almond oil.

Table 6 hereinbelow presents the accumulated concentrations of limonene diffused from the gum karaya-Valencia orange essential oil patches. For the first 12 h, limonene transferal was not detected. At 24 h, an accumulated concentration of 0.31 mg L−1 was detected. After 48 h, a 4.3-fold higher concentration was observed.

TABLE 6
Accumulated concentrations of limonene diffused from
gum karaya-Valencia orange essential oil patches^{z}

| Elapsed time | d-Limonene |
|---|---|
| (h) | (mg L⁻¹) |
| 0 to 12 | 0.00 ± 0.00^{a} |
| 24 | 0.31 ± 0.07^{b} |
| 36 | 0.68 ± 0.16^{c} |
| 48 | 1.34 ± 0.02^{d} |

^{z}Results are expressed as mean ± standard error.
^{a,b}Different superscript letters within a column indicate a statistically significant difference at P < 0.05.

The above reported flux results for linalool and camphor over time are presented in Tables 5 and 6, where it can be seen that the higher the flux, the higher the accumulated concentration of any of the transferred ingredients. In comparison to linalyl acetate and camphor, limonene exhibited the lowest flux, and it is therefore no surprise that the accumulated concentration was low and apparently of the same magnitude as that detected for a patch that transfers linalyl acetate. It is also important to note that when the skin was smeared with an almond-Valencia oil mixture, no limonene was detected.

The transferal ability of several constituents through the skin, is theoretically dependent on three factors: the molecular weight of the particular constituent, its relative solubility in water and the logarithm of its partition coefficient (Log P). The constituents of lavender and Valencia orange essential oils that successfully penetrated the skin had similar molecular masses, ranging between 136 and 196 Da. However, their solubilities differed, being relatively high for linalool and camphor and relatively low for linalyl acetate and limonene. Furthermore, Log P of linalool and camphor was about 3 and for linalyl acetate and limonene it was about 4. The optimal Log P for the transfer of ingredients with a molecular mass of about 250 Da through the skin is between 2 and 3. The Log P value of linalool and camphor was therefore optimal for delivery and thus their transfer was higher. Furthermore, both limonene and linalyl acetate are lipophilic materials with lower solubility in water, and they are therefore able to pass through the lipophilic epidermal skin layer, but then are trapped in the dermis. On the other hand, linalool and camphor are moderately lipophilic and can be soluble in water. They are able to pass through the epidermis as well as the dermis. These results explain the relatively good transfer of the linalool and camphor in comparison to linalyl acetate and d-limonene and are supported by the estimated diffusion rates.

Table 7 hereinbelow summarizes the contents (in percentage of the initial amount of entrapped essential oil) of linalool, linalyl acetate and camphor that were transferred through the skin during 24 h of treatment. The lowest proportion of transfer for these ingredients was about 0.01% (for linalyl acetate), and the highest proportion was 3.93% (linalool). It is thus clear that the assumption of the entrapped essential oil serving as an “infinite” dose is indeed correct, since less than 5% of the content diffused from the patch or the mixture through the skin during the entire experiment. Furthermore, these results are in line with the diffusion rates presented earlier.

TABLE 7
Percentages of diffused linalool, linalyl acetate and camphor vs. elapsed time from Lavandula
angustifolia essential oil patches (“Lavandula patch”) and a mixture of almond oil-
Lavandula angustifolia essential oil rubbed into the skin (“Almond-lavandula mixture”)^{z}

| | % Diffused linalool | | % Diffused linalyl acetate | | % Diffused camphor | |
|---|---|---|---|---|---|---|
| | | Almond- | | Almond- | | Almond- |
| | Lavandula | lavandula | Lavandula | lavandula | Lavandula | lavandula |
| | patch | mixture | patch | mixture | patch | mixture |
| 0 | 0.00^{a} | 0.00^{a} | 0.00^{a} | 0.00^{a} | 0.00^{a} | 0.00^{a} |
| 6 | 0.73 ± 0.16^{b} | 1.29 ± 1.53^{a} | 0.008 ± 0.011^{a} | 0.107 ± 0.150^{a} | 0.70 ± 0.04^{b} | 0.01 ± 0.01^{a} |
| 12 | 1.53 ± 0.03^{c} | 2.01 ± 2.09^{a} | 0.013 ± 0.018^{a} | 0.153 ± 0.216^{a} | 1.02 ± 0.01^{c} | 1.15 ± 1.26^{a} |
| 24 | 2.74 ± 0.31^{d} | 3.93 ± 0.01^{a} | 0.009 ± 0.001^{a} | 0.246 ± 0.344^{a} | 2.10 ± 0.32^{d} | 2.28 ± 0.08^{a} |

^{z}Results are expressed as mean ± standard error.
^{a,b}Different superscript letters within a column indicate a statistically significant difference at P < 0.05.

It thus appears from the ex-vivo experiments that terpenoids (linalool, linalyl acetate and camphor) have a better ability to penetrate the skin barrier than terpene (limonene). Note that rubbing the skin with a mixture of almond-essential oil is not actually identical to conventional massage, since systematic heating of the skin was not performed. The rate of transfer of lavender oil from the patch was similar to that from the mixture of almond-lavender essential oil, although with the former, better repetition of results was obtained. As for the Valencia essential oil, limonene transfer was only detected in the case of the patch.

### Example 6

### In-Vivo Experiments

Male Sprague-Dawley rats, weighing 300-350 g, were used in the studies (Harlan, Rehovot, Israel). The experimental protocols were approved by the Hebrew University of Jerusalem Committee on the Use and Care of Animals. Rats were maintained under specified-pathogen-free (SPF) conditions and were allowed to acclimate to the environment for at least 7 days before their use in the study. Rats were anesthetized and the fur on their stomach shaved prior to exposure. Gum karaya patches with and without lavender essential oil were applied to the rat's stomach skin. Each rat was housed in an individual cage in order to prevent oral contamination of the essential oil by one rat eating another's patch. Blood samples (0.5 ml) were taken at 0, 12, 24, 36, 48 and 60 h from a nick at the tip of tail to determine contents of essential-oil components. Rats were restrained and the tail was gently massaged to facilitate blood collection. Blood samples were collected in vials containing 0.1 ml heparin (100 kU, Sigma). A 10-μl volume of aqueous linalyl isovalerate stock solution was added as an internal standard to each blood sample and samples were stored at −80° C. After 60 h, patches were removed and the application site was wiped with 70% alcohol. Animals were sacrificed and the patch of stomach skin to which the essential oil patch had been applied was removed and minced into small pieces. The skin pieces were placed in 1 ml of ethanol containing linalyl isovalerate as the internal standard and sonicated for 30 min after heating to 50° C. A 10-μl aliquot was then injected straight into the gas chromatograph. A urine sample (0.5 ml) was obtained from the bladder immediately after sacrifice. Urine samples were similarly analyzed by gas chromatography (GC).

Blood (0.5 ml) or phosphate buffer (0.5 ml) was collected at t=0, 12, 24, 36, 48 and 60 h. Blood samples were placed in Eppendorf heparin-coated glass vials (Agilent) by heparin-washed syringe, which were sealed with septa and screw caps. Each vial contained 100 μl of heparin and blood samples were frozen at −80° C. for storage. Prior to analysis, the blood or phosphate buffer was thawed to room temperature, a 10-μl volume of internal standards (6.5 mg linalyl isovalerate and 6.5 mg geranyl formate in 25 ml acetonitrile) was added to the vial and then the mixture was vortexed. The vials were tightly capped using a clear cap with septum. The septa were pre-punctured immediately before analysis using a needle to facilitate the insertion of the fiber sheath. The fiber sheath was inserted through the septum, and positioned to expose the fiber in the center of the headspace. The vial was placed on a heating block to maintain temperature at a constant 50° C. and the solid-phase microextraction (SPME) assembly was clamped securely. Absorption time was 30 min. No stirring was required. After absorption, the SPME fiber sheath was immediately transferred to the GC injector for desorption for 5 min and GC analysis.

Calibration Curves:

Linalyl isovalerate and geranyl formate were tested for their suitability as internal standards for linalool and linalyl acetate using headspace (HS)-SPME. Linalyl isovalerate was chosen as the most appropriate internal standard on the basis of area formed under the peak. A stock solution of linalyl isovalerate in acetonitrile (25 mg L⁻¹) was further diluted with acetonitrile to produce an aqueous solution of linalyl isovalerate (5 mg L⁻¹). A 10-μl volume of the aqueous linalyl isovalerate stock solution was added as the internal standard to each sample. Standard was prepared from 0.5 ml of blank blood, 10 μl internal standard (0.0026 mg linalyl isovalerate in 25 ml acetonitrile) and an appropriate dilution of lavender oil in 10 μl acetonitrile. Lavender oil was first dissolved in acetonitrile (625 mg L⁻¹) and further diluted with acetonitrile to make intermediate standards (0.5, 1, 5, 25 and 125 mg L⁻¹). The calibration curves were plotted using the peak area ratio of linalool (the main constituent of lavender oil) and linalyl isovalerate vs. linalool concentration. A standard stock solution of lavender oil (625 mg L⁻¹) was prepared in acetonitrile. Calibration standards for determination of linear dynamic range were prepared by serial dilutions with acetonitrile. For quantitative analysis of lavender oil in the blood, working standard solutions containing 0.5, 1, 5, 25 and 125 mg L⁻¹ of lavender were prepared by dilution of the 625 mg L⁻¹ solution with acetonitrile. First, 0.5 ml of a blood sample and 10 μl of linalyl isovalerate as an internal standard were placed into a 2-ml vial, and sealed rapidly with septum and cap. The vial was then heated at 50° C. for 30 min with an aluminium block heater (Thermo Scientific, Waltham, Mass.). The needle of the SPME device, containing an extraction fiber, was passed through the septum and the extraction fiber was exposed to the gas phase for 30 min. The fiber was drawn back into the needle and removed from the vial, then inserted into the injection port. The compounds absorbed on the fiber were detached and analyzed by exposing the fiber for 5 min in the injection port.

GC Conditions:

GC was performed on an Agilent 7890A gas chromatograph equipped with a split/splitless capillary injector, flame ionization detection (FID) system and GC Chem Station (Version B.03.01; Agilent Technologies 2007). Chromatography was carried out on a WAX or HP-5 column (30 m×0.32 mm×0.25 μm). The GC operating conditions were: splitless or split 1:40 injector 240° C., oven 50° C. for 1 min and then 10° C. min⁻¹ to 160° C., then 25° C. min⁻¹ to 240° C. and held for 6 min; the carrier gas was hydrogen.

Statistical Analysis:

Statistical analyses were conducted with JMP software (SAS Institute 2007, Cary, N.C.), including ANOVA and Tukey-Kramer Honestly Significant Difference test for comparisons of means. P≦0.05 was considered significant.

A. Standards and Calibration Curves

Two standards were checked for their suitability to this study: linalyl isovalerate and geranyl formate. GC of geranyl formate in the blood revealed a few peaks that were identical to linalool and linalyl acetate, the constituents of lavender essential oil, and they therefore could not be used with this essential oil. Nevertheless, GC-mass spectrometry (MS) showed that geranyl formate decomposes in the blood to cis-geraniol, geranial, amyl vinyl alcohol and citronella, which are not components of lavender essential oil. GC of linalyl isovalerate in the blood revealed a peak with the same retention time as the linalool in the lavender essential oil. GC-MS showed that linalyl isovalerate decomposes in the blood to linalool and isovaleric acid. 1-Octen-3-ol, d-limonene, β-myrcene and β-cis-ocimene, presumed products of the chemical reaction of linalool, were also identified/detected. Thus, although it is not possible to quantify linalool since the lavender essential oil may not be its only source, it is possible to quantify other components of lavender, and linalyl isovalerate was chosen to serve as the standard in the blood.

Two GC columns, WAX and HP-5, were tested for their suitability to this study. Components were identified by GC-MS with these columns. The main differences between the tests were that the flushing gas was hydrogen in GC and helium in GC-MS. As a result, a small difference in retention times between GC and GC-MS results was detected, although the order and sizes of the peaks remained constant. Constituents were identified by comparing the GC-MS library to the list of essential-oil constituents provided by its manufacturer/supplier. With the WAX column we used split injection (1:40), meaning that only part of the absorbed sample reaches the column. The observed retention times for camphor, linalool and linalyl acetate were 9.475, 9.763 and 9.913 min, respectively. The respective correlation coefficients were 0.992, 0.999, and 0.998, demonstrating a highly linear correlation between the chromatogram peak area and the concentration of the constituent in mg L⁻¹. This calibration was used in both the preliminary experiment and in further in-vivo experiments. Using the HP-5 column and splitless injection, i.e. the whole absorbed sample reaches the column, we could identify more constituents than with the WAX column. The observed retention times for linalool, camphor, borneol, α-terpineol, linalyl acetate and β-farnesene were 7.777, 8.522, 8.807, 9.130, 9.934, and 12.463 min, respectively. The respective correlation coefficients were 0.999, 0.979, 0.983, 1.000, 0.999 and 0.999, again demonstrating a high linear correlation between chromatogram peak area and constituent concentration. This calibration was used in our further in-vivo experiments.

B. Preliminary In-Vivo Experiment

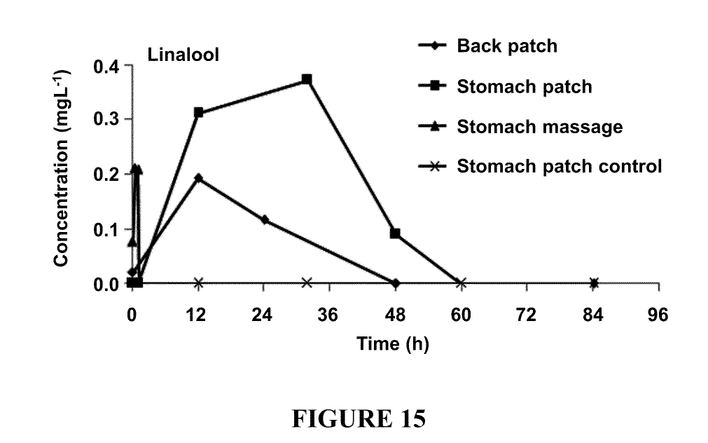

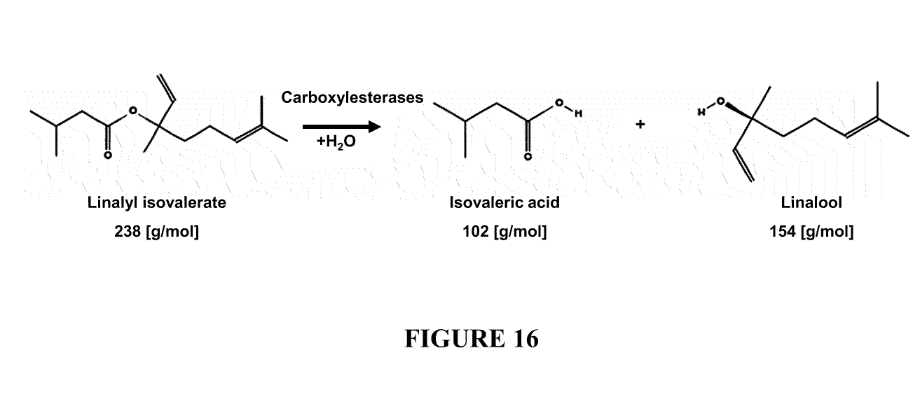

Skin is not homogeneous in composition, roughness, topography or the amount of hair follicles per unit area. Since these factors might influence both patch adhesion to the skin and the transfer of essential-oil constituents through it, transfer of essential-oil constituents was first tested with patches containing 7.5% lavender essential oil that were adhered to both the shaved back and abdomen skin of rat. In addition, direct smearing (massage-like simulation) of a mixture of almond oil and lavender oil (same concentration as in the patch) to the same areas was carried out. Blood analysis was conducted by GC using the WAX column. FIG. 15 demonstrates the accumulated concentration of linalool (mg L⁻¹) identified in the blood samples vs. time elapsed from the instant when the patch was adhered to the skin or skin massage was conducted. When a patch without lavender oil (blank) was adhered to the skin, the detected linalool concentration vs. time (FIG. 15) was found to equal 0.11±0.03 mg L⁻¹. This amount is not negligible and its source might be the decomposition of the internal standard, linalyl isovalerate, in the blood (FIG. 16). To locate and eliminate other sources of linalool, a blank patch, the food consumed by the rats, as well as the air and water to which the animals were exposed were studied. No traces of linalool were found. For all treatments, i.e. back patch, patch adhered to the abdomen skin or skin after massage, the concentration of linalool was higher than in the control group. These higher values are thought to be related to the direct transfer of linalool through the skin, as well as/or instead of the decomposition of linalyl acetate, which is one of the ingredients of lavender essential oil (FIG. 16). Such a reaction can occur as a result of enzyme (esterase) activity in the blood or of the blood's oxidation-reduction ability. For a patch adhered on the abdomen side (opposite to back), higher levels of linalool were observed, possibly due to differences in the roughness of the skin in these two locations. For 80 min after having rubbed the skin with the almond-essential oil mixture, linalool was evident in the blood. The highest concentration (0.32 mg L⁻¹) was measured 30 min after the application, but it was still lower than the highest value (0.48 mg L⁻¹) achieved 36 h after a patch had been adhered to the abdomen area. Very limited information is available on the fate of essential oils after application to an organism. The most relevant information could be found was that traces of linalyl acetate, as well as linalool, can be detected in the blood of mice exposed to pure linalyl acetate via breathing (Jirovetz et al., 2004).

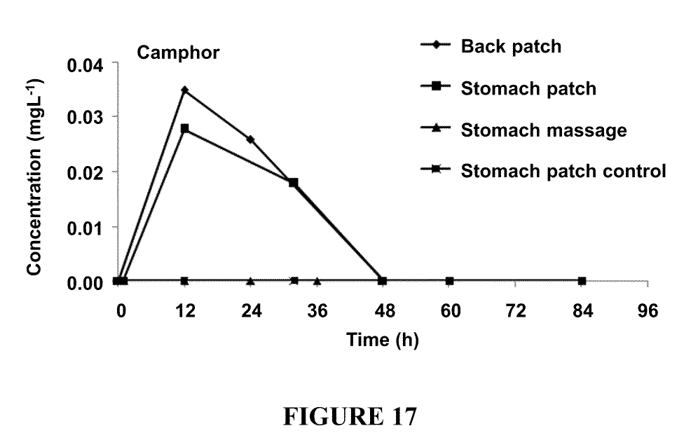

Similar values of camphor (FIG. 17) were detected following application of a lavender oil patch on the back and abdomen. No levels of camphor were detected with the blank patch or after rubbing with the oil mixture. In the blood of rats with an abdomen-adhered patch, more linalyl acetate was detected than in the blood of rats with a back patch. No linalyl acetate was detected when the blank patch or massage was used. In a collected urine sample, 0.01265 mg L⁻¹ linalool and 0.00903 mg L⁻¹ linalyl acetate were detected. In the literature, an older report was located on other metabolites of linalool that can be identified in the urine, such as 8-hydroxy-linalol and 8-carboxy-linalol, but an analysis of all components in the urine was beyond the scope of this study. In addition, no traces of camphor were detected in the urine (note that the urine analysis was performed 84 h after the simulated massage).

Due to the observed better transfer of lavender essential-oil components through the abdomen skin, we decided to focus on patches adhered to the stomach skin and to investigate the transfer of the essential-oil components using two different GC columns, WAX and HP-5.

C. In-Vivo Experiment with WAX Column

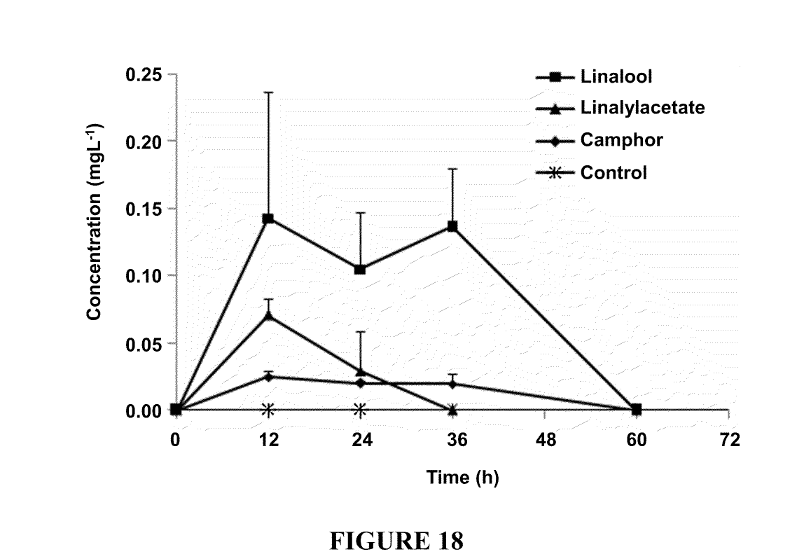

Patches that included lavender essential oil were adhered to the abdomen skin for 60 h. Patches without entrapped oil served as blanks. Patches remained glued to the skin and blood samples were taken every 12 h and tested by GC with a WAX column (split injection mode, 1:40). FIG. 18 presents the concentrations of linalool, linalyl acetate and camphor in the blood. A similar pattern was observed for linalool and camphor. Between 12 and 36 h, a similar, non-significant change in levels was detected. Later on, a significant decrease in these levels were observed. After 60 h, there were no traces of these constituents in the blood. In general, camphor reached a level between 0.020 and 0.025 mg L⁻¹ and linalool between 0.105 and 0.114 mg L⁻¹. In the blank, camphor was not detected, whereas a constant level of linalool was related to the decomposition of the standard linalyl acetate. FIG. 18 was redrawn after subtracting the 0.11% linalool found in control blood samples from all numerical values, and therefore the control is regarded as “zero”. An increase in linalyl acetate concentration was observed after 12 h, followed by a decrease during the next 24 h.

D. In-Vivo Experiment with HP-5 Column

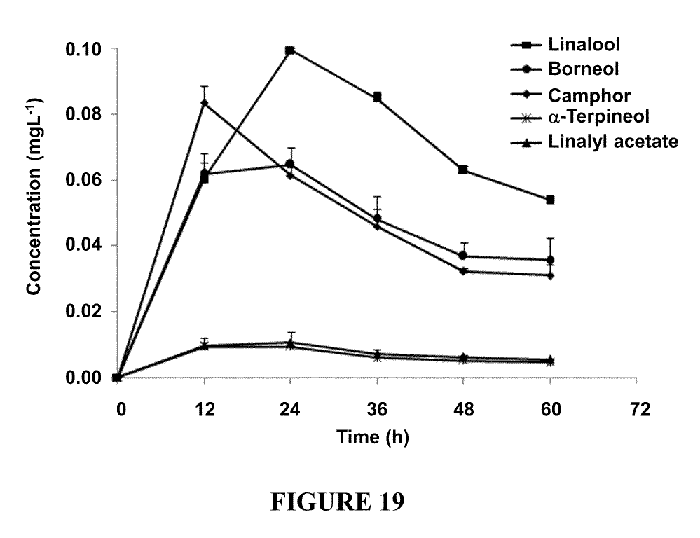

In this experiment, patches containing lavender essential oil were adhered to the abdomen area. Patches without essential oil served as blanks. FIG. 19 shows the concentrations of the constituents vs. time for 60 h. Detection under splitless conditions facilitated the detection of linalyl acetate, linalool, camphor, borneol and α-terpineol, the latter two being additional constituents detected only under these conditions. High concentrations of linalool, borneol and camphor were detected, in comparison to low levels of terpeneol and linalyl acetate. Moreover, a significant difference was detected between the levels of linalool vs. camphor and borneol 12 h into the experiment. α-Terpineol was not significantly different from linalyl acetate. Linalool reached its highest value (0.099 mg L⁻¹) after 24 h and then decreased significantly during the following 36 h to 0.053 mg L⁻¹. Camphor and borneol reached their highest level after 12 h (0.083 and 0.061 mg L⁻¹, respectively). Borneol level decreased during the next 12 h to the level of camphor, which stayed constant, and then both constituents decreased to 0.035 and 0.031 mg L⁻¹, respectively, after 60 h.

α-Terpeneol level increased after 12 h to a level of 0.005 to 0.009 mg L⁻¹, where it stayed for the next 48 h. Linalyl acetate showed a similar pattern, increasing to between 0.006 and 0.009 mg L⁻¹ after 12 h. It is important to note that no component of the essential oil fell to a value of zero after 60 h, in contrast to the values obtained under split-injection conditions. In conclusion, the chemical analyses performed with WAX and HP-5 columns yielded different results; however, it is important to note that different analyses will yield slightly different results since in general, there is a problem in determining terpene levels in the blood due to their tendency to undergo isomerization, or decomposition in weak acidic solutions such as blood; moreover, terpenes can serve as enhancers for other terpenes and thus their presence and level might change results relative to mixtures in which the composition is different.

E. Extraction of Lavender Essential Oil from the Skin

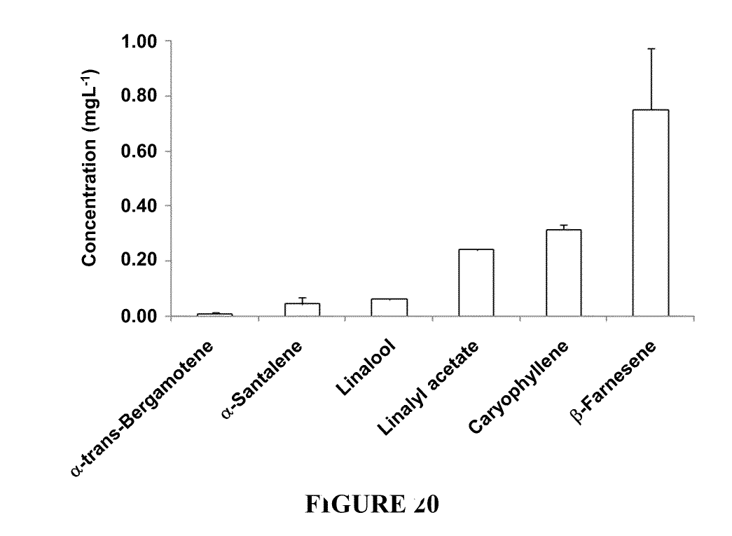

Skin to which patches with or without lavender essential oil were adhered were sampled for extraction. Average skin weight was about 3.1×10⁻⁴ kg and its average thickness was about 965 μm. When a control patch was adhered to the skin, no constituents of lavender essential oil were detected. When the essential oil-containing patch was adhered to the skin, the first interesting phenomenon was that in contrast to that which occurred in the blood, the internal standard linalyl isovalerate did not decompose to its constituents linalool and linalyl acetate. FIG. 20 demonstrates the observed concentrations of different constituents that were extracted from the skin. The high concentration of linalyl acetate in comparison to linalool, 0.241 mg L⁻¹ vs. 0.062 mg L⁻¹, respectively, was opposite to the situation observed in the blood (0.009 mg L⁻¹ for linalyl acetate vs. 0.099 mg L⁻¹ for linalool). In addition, other constituents of lavender essential oil were identified, such as α-santalene, caryophyllene, β-farnesene and α-trans-bergamotene, which were not identified in the blood.

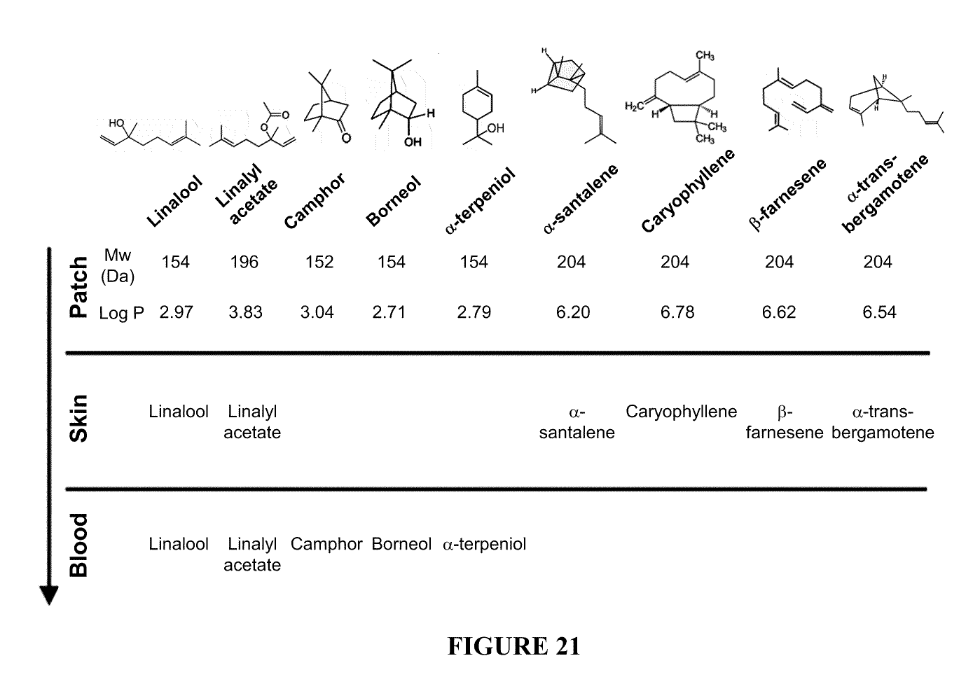

Based on both the ex-vivo and in-vivo experiments, a hypothetical model can be proposed for the mechanism underlying the delivery of essential-oil components through the skin, from either patches or massaging with an oil mixture (FIG. 21). The essential oil cannot be regarded as one component, but as a mixture of ingredients that may or may not penetrate the skin, depending on many factors: the ingredient's molecular weight, its solubilization in the skin components, the log of the partition coefficient, the temperature of the skin, the energy invested in massaging the skin when applied, and other parameters such as the adhesion of the patch to the skin, its topography, and the physical and chemical properties of the patches. In fact, the picture is much more complicated. Ingredients of essential oils may remain in the patch, be entrapped in the skin with no further penetration, or be less prone to evaporation, making them unidentifiable by the SPME method. It should be noted that the extracted solution was injected directly into the GC, in contrast to the SPME method used for the blood analysis. Those ingredients that did penetrate the blood were detected there as is or decomposed, similar to the situation for constituents found in the urine.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Ben-Zion et al. 2008 J Adhes Sci Technol, 22:205-16.

Bergman B, Lowhagen G B, Mobacken H. 1982. Irritant skin reaction to urostomal adhesives. Urol. Research 10:153-5.

Chen J L, and Cyr G N. 1970. Compositions producing adhesion through hydration in: Manly R S, ed. Adhesion in Biological Systems. NY: Academic Press; pg 163-81.

Goulding T M., 1994. Pressure-sensitive adhesives. In: Pizzi A, ed. Handbook of Adhesive Technology. N.Y: Marcel Dekker Inc., pgs 549-64.

Hammond F H. 1989. Tack. In: Satas D, ed. Handbook of Pressure-Sensitive Adhesives Technology. NY: Van Nostrand Reinhold, pgs 32-9.

Tan H S, Pfister W R. 1999. Pressure-sensitive adhesives for transdermal drug delivery systems. Pharmaceutical Science & Technology Today, 2:60-9.

1. A dermal patch comprising a bioadhesive composition comprising at least one natural polysaccharide exudate, at least one essential oil, a co-solvent, an emulsifier, and water, substantially devoid of synthetic pressure sensitive adhesives.

2. The patch according to claim 1, wherein the water content of the patch is above 5%, preferably above 10%.

3. The patch according to claim 1, wherein the polysaccharide exudate is a plant exudate selected from the group consisting of Sterculia foetida, Bauhinia variegata, Buchnania lanzan, Terminalia crenulata, Terminalia catappa, Terminalia belerica and gum karaya.

4. The patch according to claim 1, wherein the co-solvent is propylene glycol.

5. The patch according to claim 1, wherein the essential oil is present at a concentration of 1-10% (w/w), preferably at a concentration of 2.5-10%.

6. The patch of claim 1 comprising a mixture of essential oils.

7. The patch according to claim 1, further comprising a removable porous or non-porous backing layer or liner covering the surface intended for contact with the skin.

8. The patch according to claim 1, further comprising a porous or non-porous cover layer on the surface opposite the side intended for contact with the skin.

9. The patch of claim 1 further comprising at least one excipient selected from a filler, a penetration enhancer, and a viscosity modifier.

10. The patch of claim 10 comprising an inert filler.

11. The dermal patch of claim 1 consisting essentially of at least one natural polysaccharide exudate, at least one essential oil, an emulsifier, water and at least one co-solvent.

12. The patch of claim 1 wherein the maximal tack force required to separate the patch from a skin model is in the range of 0.5 to 4 N.

13. The patch of claim 1 wherein the degree of elasticity is at least 60%.

14. The patch of claim 10, wherein the degree of elasticity is at least 55%.

15. The patch according to claim 1 wherein the patch is suitable for multiple applications to the skin.

16. An adhesive dermal patch comprising at least one hydrophilic polymer derived from a natural exudate, containing above 5% (w/w) water, substantially devoid of synthetic pressure sensitive adhesives.

17. The patch according to claim 16, wherein the dermal patch further comprises an active agent selected from a therapeutic agent and a cosmetic agent.

18. A method for transdermal delivery to a subject of at least one essential oil comprising applying to the skin of the subject a dermal patch comprising a bioadhesive composition comprising at least one natural polysaccharide exudate, at least one essential oil, a co-solvent, an emulsifier, and water, wherein the patch is substantially devoid of synthetic pressure sensitive adhesives.

19. The method of claim 17 wherein the patch is effective in delivery of the at least one essential oil over a period of at least 24 hrs.

20. The method of claim 19 where in the patch is effective in delivery of the essential oil over a period of at least 2-3 days.
